(19) United States
(12) Patent Application Publication
(10) Pub. No.: US 2009/0215965 A1
(43) Pub. Date: Aug. 27, 2009

(54) POLYMERISATION USING CHAIN TRANSFER AGENTS

(75) Inventor: Sebastian Perrier, Haxby (GB)

Correspondence Address: WIGGIN AND DANA LLP;ATTENTION: PATENT DOCKETING, ONE CENTURY TOWER, P.O. BOX 1832, NEW HAVEN, CT, 06508-1832, US

(73) Assignee: The University of Leeds, Yorkshire (GB)

(21) Appl. No.: 12/420,361

(22) Filed: Apr. 8, 2009

Related U.S. Application Data
(63) Continuation of application No. 10/583,809, filed on Jun. 7, 2007, now abandoned.

(30) Foreign Application Priority Data
Dec. 23, 2003 (GB) 0329815.5
Sep. 7, 2004 (GB) 0419835.4

(51) Int. Cl. C08F 8/00; C07C 327/16; C07C 313/00

(52) U.S. Cl. 525/328.4; 558/230; 558/62

(57) ABSTRACT

The invention provides a process for synthesising functionalised chain transfer polymers of Formula (1) or Formula (2) using thiocarbonyl thio compounds as chain transfer agents. Why R1 is a moiety comprising a functional group; Q is obtained from an olefinically unsaturated monomer; R′ is selected from the group consisting of alkyl, substituted alkyl, alkoxy, substituted alkoxy, an aromatic saturated or unsaturated carbocyclic or heterocyclic ring, optionally substituted with one or more substituents, amino alkyl, cyanoalkyl, hydroxylalkyl, saturated and unsaturated amido; an organometallic species, a polymer chain and any of the foregoing substituted with one or more CN or OH groups; q=an integer of at least 2; p=an integer of at least 1. Chain transfer agents and polymers produced by the method are also provided.

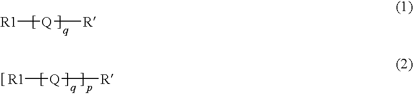

This invention relates to a process for synthesizing polymers using a thiocarbonyl thio compound as a chain transfer agent. The invention also relates to functionalized polymers produced by the process and to thiocarbonyl thio intermediates that may be employed in the process.

A controlled process is required in a polymer or copolymer synthesis to achieve a product with properties such as a desired molecular weight and a narrow weight distribution, or polydispersity. Polymers with a narrow molecular weight distribution can exhibit substantially different behaviour and properties to polymers prepared by conventional means. Living radical polymerizations (sometimes referred to as controlled free radical polymerizations) provide a maximum degree of control for the synthesis of polymers with predictable and well-defined structures. Recently, living radical polymerization has been shown to be a viable technique to prepare a large diversity of block copolymers.

The characteristics of a living polymerization include: polymerization proceeding until all monomer is consumed, number average molecular weight as a linear function of conversion, molecular weight control by the stoichiometry of the reaction, and block copolymer preparation by sequential monomer addition.

It has been stated that living polymerization to give polymers of a low molecular weight distribution requires the absence of chain transfer and termination reactions. In a living polymerization, the only “allowed” elementary reactions are initiation and propagation. These take place uniformly with respect to all growing polymer chains. However, it has also been shown that if the chain transfer process is reversible, polymerization can possess most of the characteristics of a living polymerization.

It has been found that the reversible addition-fragmentation chain transfer (RAFT) process suppresses termination reactions through the addition of a suitable thiocarbonyl thio compound to an otherwise conventional free radical polymerization. Control in such a RAFT process is thought to be achieved through a degenerative chain transfer mechanism in which a propagating radical reacts with the thiocarbonyl thio compound to produce an intermediate radical species. This process decreases the number of free radicals available for termination reactions that require two free radicals. The use and mechanism of control agents for free radical polymerization is now generally known, see for example U.S. Pat. No. 6,153,705, WO98/01478, WO99/35177, WO99/31144, and WO98/58974. Despite this knowledge, no successful commercialization of a polymerization process has occurred using these agents. There is a need for new agents which may lead to a commercializable process.

In addition, the previously known control agents have limited uses. Although suggested to be universally useful, those of skill in the art appreciate that a particular chain transfer agent is useful for the control of particular monomers and monomer mixtures. The polymerization conditions under which particular transfer agents are useful are generally not well known.

Thus, a need exists for a family of related control agents that can be easily synthesized and modified so that a claim transfer may be readily available for polymerizing desired monomers under commercially acceptable conditions, which include recycling of the control agent and production of readily usable polymers. From a process point of view, an agent that can be recovered for the process is needed. In addition polymers obtained by the previous techniques present a thiocarbonyl thio end group. There is a need for a technique to produce polymers with a specific end-group. This also would have the advantage of removing potentially toxic thio-containing moieties from the polymer and allows recycling of the control agent. Additionally, there is a strong need in the industry to make block copolymers.

According to a first aspect of the present invention there is provided a method of making a functionalised polymer of Formula (1) or Formula (2).

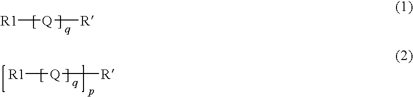

comprising the steps of:
reacting a thiocarbonyl thio compound of Formula (3) or Formula (4);

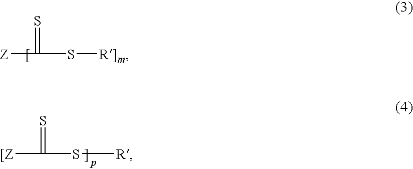

an olefinically unsaturated monomer (O), and a first source of free radical to form a polymer of Formula (6) or Formula (7);

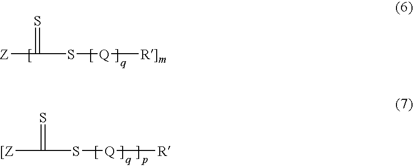

and subsequently contacting the polymer of Formula (6) or Formula (7) with a second source of free radicals, the second source of free radicals comprising a radically transferable functional moiety R1, to form a polymer of Formula (1) or Formula (2) and a compound of Formula (3) or Formula (4);
wherein:
R1 is a moiety comprising a functional group
R′ is selected from the group consisting of alkyl, substituted alkyl, alkoxy, substituted alkoxy, an aromatic saturated or unsaturated carbocyclic or heterocyclic ring, optionally substituted with one or more substituents, amino alkyl, cyanoalkyl, hydroxylalkyl, saturated and unsaturated amido; an organometallic species, a polymer chain and any of the foregoing substituted with one or more CN or OH groups; preferably the group contains from 2 to 10 carbon atoms.
Z is selected from a solid support, Z comprises a linker attached to a solid support, or Z is a group selected from a straight or branched chain, substituted or non substituted C₁ to C₂₀ alkyl (especially a C₁ to C₄ alkyl such as methyl or ethyl); optionally substituted aryl, e.g. phenyl, substituted phenyl; phenyl covalently bonded to a polymer; optionally substituted heterocyclyl, substituted or non-substituted C₁ to C₂₀ (especially C₁ to C₄) alkoxy, optionally substituted alkyl thio, thioalkoxyl (optionally substituted with a polymer); substituted or non-substituted benzyl (optionally substituted with a solid support), optionally substituted aryl oxycarbonyl (—COOR″), carboxy (—COOH), optionally substituted ocyloxy (—O₂CR″), optionally substituted acyloxy (—CO₂CR″), optionally substituted carbornyl (—CONR₁₂), cyano (—CN), dialkyl- or diaryl phosphonato (—P(═OR″Z), dialkyl- or diaryl-phosphinato [—P(═O)R″Z] or SCH₂CH₂ CO₂ T (where T is a solid support or a polymer); the linker may optionally comprise a straight or branched chain, substituted or non substituted C₁ to C₂₀ alkyl (especially a C₁ to C₄ alkyl such as methyl or ethyl); phenyl, substituted phenyl; phenyl covalently banded to a polymer; substituted or non-substituted C₁ to C₂₀ (especially C₁ to C₄) alkoxy, thioalkoxyl (optionally substituted with a polymer); substituted or non-substituted benzyl; most preferably z is a solid support or a linker attached to a solid support.
R″ is selected from the group consisting of optionally substituted C₁-C₁₈ alkyl, C₂-C₁₈ alkenyl, aryl, heterocyclyl, aralkyl, alkaryl wherein the substituents are independently selected from the group that consists of epoxy, hydroxyl, alkoxy, acyl, acyloxy, carboxy (and salts), sulfonic acid (and salts), alkoxy- or aryloxycarbonyl, isocyanato, cyano, silyl. halo, and dialkylamino;
Q is at least one olefinically unsaturated monomer, optionally two or more different olefinically unsaturated monomers;
q=an integer of at least 2;
p=an integer of at least 1;
m=an integer of at least 1.

Preferably q is from 2 and 1000, most preferably at least 500;
p is preferably from 2 and 1000, preferably 100 or 500;
m is preferably from 2 to 1000, especially 800 or 500, most preferably from about 2 to 50.
Where Z is a solid support the loading of the support may be up to about 5 mmol/g.
Compounds of Formulae (3) or (4) are examples of chain transfer agents (CTAs).

Preferably the olefinically unsaturated monomer consists of vinyl monomers of Formula (5):

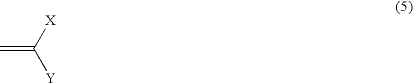

wherein X is selected from the group consisting of: hydrogen, halogen and substituted or unsubstituted C₁-C₄ alkyl, said alkyl substituents being independently selected for the group consisting of hydroxyl, alkoxy, OR″, CO₂H, CO₂R″, O₂CR″ and combinations thereof; and
wherein Y is selected from the group consisting of hydrogen, R″, CO₂H, CO₂R″, COR″, CN, CONH₂, CONHR″, CONR″₂, O₂CR″, OR″ and halogen.

The radically transferable functional moiety, R1 is, for example, an entity or fragment comprising a functional group. The functional group may be any chemical group having desired properties. These include one or more of: epoxy, oxirane, carboxylic acid, ester, hydroxyl, polyol, isocyanate, amide, amine, oxazoline, aceto acetate and carbamate groups.

In a preferred embodiment the compound of Formula (3) or Formula (4) is recovered at the end of the process. This may be, for example, precipitated or be recovered, for example, because of being attached to the preferred solid support.

Preferably the thiocarbonyl thio compound does not contain a nitrogen-nitrogen bond.

Preferably, an excess of the second source of free radicals is added. This terminates the polymerisation reaction and releases the chain transfer agent (the thio carbonyl thio compound).

Any convenient source of free radicals may be used. In a preferred aspect of the invention, the source of radical is compound capable of forming a carbon and oxygen centered radical, which is able to initiate free radical polymerization, preferably of the formula (8):

R2-W—R3 (8)

wherein R2 and R3 are independently selected from the group consisting of R′; and W may be a —N═N-bond, an —O—O— bond or a group that decomposes thermally or photolytically to form two residues containing a carbon centered radical, and at least one of R2 or R3 reacts with the polymer of Formula (6) or Formula (7) to leave the moiety comprising the functional group. The groups R′, R1, R2 and R3 may be independently the same or different.

The second initiator may be the same as the first initiator or different. Examples of the first initiator are defined later.

Preferably the first source of radical is the same as the second source of radical (i.e. the same radical initiator) and R1=R2=R3=R′ to form a telechelic polymer.

Preferred examples of the second initiator include: 2,2′-azobis(isobutyronitrile), 4,4′-azobis(4-cyanopentanoic acid, 2-(t-butylazo)-2-cyanopropane, 2,2′-azobis(isobutyramide) dihydrate, 2,2′-azobis(2-methylpropane), 2,2′-Azobis[2-(5-methyl-2-imidazolin-2-yl)propane]dihydrochloride, 2,2′-Azobis[2-(2-imidazolin-2-yl)propane]dihydrochloride, 2,2′-Azobis[2-(2-imidazolin-2-yl)propane disulfate dehydrate, 2,2′-Azobis(2-methylpropionamide)dihydrochloride, 2,2′-Azobis[N-(2-carboxyethyl)-2-methylpropionamidine]tetrahydrate, 2,2′-Azobis[2-(3,4,5,6-tetrahydropyrimidin-2-yl)propane]dihydrochloride, 2,2′-Azobis{2-[1-(2-hydroxyethyl)-2-imidazolin-2-yl]propane}dihydrochloride, 2,2′-Azobis[2-(2-imidazolin-2-yl)propane], 2,2′-Azobis{2-methyl-N-[1,1-bis(hydroxymethyl)-2-hydroxyethyl]propionamide, 2,2′-Azobis{2-methyl-N-[2-(1-hydroxybuthyl)]propionamide}, 2,2′-Azobis[2-methyl-N-(2-hydroxyethyl)propionamide], 2,2′-Azobis(4-methoxy-2,4-dimethyl valeronitrile), 2,2′-Azobis(2,4-dimethyl valeronitrile), Dimethyl 2,2′-azobis(2-methylpropionate), 2,2′-Azobis(2-methylbutyronitrile), 1,1′-Azobis(cyclohexane-1-carbonitrile), 2,2′-Azobis[N-(2-propenyl)-2-methylpropionamide], 1-[(cyano-1-methylethyl)azo]formamide, 2,2′-Azobis(N-butyl-2-methylpropionamide), 2,2′-Azobis(N-cyclohexyl-2-methylpropionamide), t-butyl peroxyacetate, t-butyl peroxybenzoate, t-butyl peroxyoctoate, t-butyl peroxyneodecanoate, t-butylperoxy isobutyrate, t-amyl peroxypivalate, t-butyl peroxypivalate, di-isopropyl peroxydicarbonate, dicyclohexyl peroxydicarbonate, dicumyl peroxide, dibenzoyl peroxide, dilauroyl peroxide, potassium peroxydisulfate, ammonium peroxydisulfate, di-t-butyl, hyponitrite, and dicumyl hyponitrite.

Functional groups give a specific property to the material or a specific chemical activity.

Preferably the specific property or chemical activity is predefined. The specific property may be a physical property, such as adding a moiety to adjust the solubility compound in a solvent. The specific property may be a chemical property, such as adding a reactive moiety to the compound.

Specific end functionalised polymers (Formula (1) or (2)) can be produced in quantitative yields. Polymers having different groups at each end may also be produced by use of appropriately selected thiocarbonyl thio compound and source of free radical. Telechelic polymers having the same end groups may be produced by using thiocarbonyl thio compound and source of free radical generating similar radical species. The present invention offers the possibility to create telechelic polymers having two functional groups at both chain ends.

The term “telechelic polymer” was proposed in 1960 by Uraneck et al. to designate relatively low molecular weight macromolecules possessing one or more, and preferably two reactive functional groups, situated at the chain ends. The functional end groups of the polymers formed therefrom, have the capacity for selective reaction to form bonds with another molecule.

The functionality of a telechelic polymer or prepolymer is equal to the number of such end groups. Telechelic polymers containing a functional group, COOH for instance, at each end are useful for synthesizing further chain extended copolymers and block copolymers. The interest in telechelic polymers resides in the fact that such polymers can be used, generally together with suitable linking agents, to carry out three important operations: (1) chain extension of short chains to long ones by means of bifunctional linking agents, (2) formation of networks by use of multifunctional linking agents, and (3) formation of (poly)block copolymers by combination of telechelics with different backbones. These concepts are of industrial importance since they form the basis of the so-called “liquid polymer” technology exemplified by the “reaction injection molding” (RIM). Interest has also been shown by the rubber industry because the formation of a rubber is based on network formation. In classical rubber technology, this is achieved by the cross-linking of long chains that show high viscosity. The classical rubber technology, therefore, requires an energy-intensive mixing operation. The use of liquid precursors, which can be end-linked to the desired network, offers not only processing advantages, but in some cases, also better properties of the endproduct. Further information about telechelic polymers and synthesis thereof can be found in “Telechelic Polymers: Synthesis and Applications” by Eric J. Goethe, CRC Press, Boca Raton, Fla., 1989.

The reaction conditions for the reactive functional acid end groups of the telechelic polymers of the present invention are generally the same as those for forming the above noted free radical polymers. The acid in the monomeric or in the polymeric form can be transformed to its derivatives in a conventional manner. For example, the ester can be made by refluxing the acid in alcohol with an acid catalyst with removal of water. Amides can be formed by heating the acid with an amine with the removal of water. 2-hydroxy-ethyl ester can be formed by directly reacting the acid with an epoxide with or without a catalyst such as triphenylphosphine or an acid like toluensulfonic acid. As illustrated below, any of the above noted monomers such as the one or more diene monomers or one or more vinyl containing monomers, can be utilized to form the telechelic monomers from the process of the present invention. Any of the above noted components, such as solvent, etc., can be utilized in the herein above stated amounts.

WO 02/26836 and US 2003/0232938 disclose nitrogen-nitrogen bond containing control agents bonded to a thiocarbonyl moiety. These may be reacted with a free radical source and an additional fragmentation agent to form a polymer with group of interest. Supported chain transfer agents are not disclosed. Furthermore the agents are not recovered.

Many of the free non-supported chain transfer agents used in the current invention have advantages over those in the prior art, such as lower boiling points allowing milder reaction conditions for recycling the chain transfer agents. Furthermore, preferably Z=phenyl. This allows improved control over molecular weight and polydispersities. Methacrylates and their derivatives may be polymerised with more control.

The method of the present invention provides advantages over previously known methods of polymerization using chain transfer agent: The process reported in this invention produces (co)polymers with low polydispersities and a wide range of specific functionalities at the polymeric chain-end. Also, following completion of reaction, the thiocarbonyl thio intermediate may be recovered. Addition of a further quantity of monomer may lead to reuse of the thiocarbonyl thio intermediate to produce polymer of similar molecular weight, so that the amount of thiocarbonyl thio intermediate required to produce a particular quantity of polymer is substantially reduced. Alternatively, the intermediate may be separated from the polymer in the reaction mixture and isolated for reuse in the same or different process. This reduces environmental problems caused by the need to produce and dispose of a large quantity of the dithio intermediates. The dithio intermediate may be separated by distillation or sublimation. Amphiphilic intermediates may be isolated by phase separation. In a preferred aspect of the invention Z is a solid support or is not a solid support. Use of a solid support facilitates separation of the resultant polymer from the solid supported thiocarbonyl thio compound.

The compounds of Formula (3) or Formula (4) attached to a polymer or most preferably a solid support have advantages. Firstly, they are easier to recover, thus removing potentially toxic thio compounds from the product Secondly, they lead to products with lower amounts of dead chains than those of the prior art.

Products synthesized via the previously reported techniques (RAFT, MADIX, Symyx's system, etc) include a low amount of ‘terminated chains’ (‘dead’ chains), arising from the termination reaction due to the presence of a source of free radical to initiate polymerization.

These dead chains will have an uncontrolled molecular weight which will increase the overall PDI of the system. An additional problem arising from the presence of dead chains in the system is encountered during the production of block copolymers. Block copolymers can be produced by the sequential addition of a different types of monomers, after the first batch of monomer has fully reacted. Upon addition of a second batch of monomer, the chains will be reactivated and further polymerised. Unfortunately, dead chain will not be able to re-initiate the second batch of monomer, which will lead to a mixture of block copolymer with homopolymers. However, when using a solid supported CTA, only the ‘living chains’ are attached to the support, and the dead chains can be filtered out. After filtration, the chains attached to the support should have low PDI, and all chains should be able to reinitiate polymerization, leading to block copolymer with no homopolymers side-products.

Accordingly the invention also provides a method of producing a block copolymer comprising reacting a first unsaturated monomer by a method according to any one of claims 1 to 20, wherein the thiocarbonyl thio compound of Formula (3) is supported on a solid support, recovering polymer attached to the solid support, and then reacting the recovered polymer by the method of any one of claims 1 to 20 with a second unsaturated monomer to form a block copolymer.

Alternatively, Z comprises, more preferably consists of, a linker attached to a solid support, the linker attaching the thiocarboxylthio moiety to the solid support.

The solid support may be organic or inorganic such as Wang resin, Merrifield resin, silica (e.g. silica gel), alumina or magnetised beads. Such supports may be derivatized by techniques generally known in the art to attach the thiocarboxylthio moiety or the linker.

The polymer may be a conventional condensation polymer such as a polyester (e.g. polycaprolactone, poly(ethylene terephthalate), polycarbonates, polyalkylene oxides (e.g. polyethylene oxide), nylons, polyurethanes or addition polymers formed by coordination polymerisation (e.g. polyethylene), radical polymerisation (e.g. poly(meth)acrylates), and polystyrenics or anionic polymerisation (e.g. polystyrene or polybutadiene).

The alkyl may comprise one or more aromatic groups as part of the alkyl chain.

Preferably, Z contains 1, 2, 3, 4, 5, 6, 7, 8, 9, 10, 11, 12, 13, 14, 15, 16, 17, 18, 19 or 20 carbon atoms.

Organometallic species preferably means a moiety containing one or more metal atoms of groups III and IV of the periodic table and transition and organic ligans, e.g. Si(X)₃, Ge(X)₃, SnX₃ which provide radical leaving groups. Where X is substituted or non-substituted methanine nitrogen or a conjugating group.

Scheme 1 illustrates a general process wherein a thiocarbonyl thio compound (1) reacts with a vinyl monomer (2) to form an intermediate polymer in the presence of a first free radical source. Addition of a second radical source R—W—R (3) to this intermediate polymer leads to the formation of a polymer with R as end-groups (4) and allow the recovery of the initial thiocarbonyl thio compound (I).

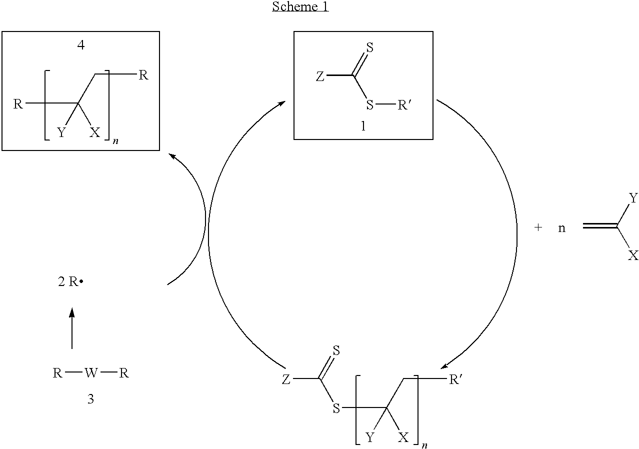

Preferred groups Z are selected from the group consisting of:
methyl, ethyl, other C₁-C₄ alkyl, [methylene covalently bonded to a polymer, methylene covalently bonded to a solid support T], phenyl, substituted phenyl, phenyl covalently bonded to a polymer, preferably phenyl covalently bonded to a solid support T, alkoxy, substituted alkoxy, thioalkoxy, substituted thioalkoxy, alkoxy or thio alkoxy substituted with a polymer, preferably thioalkoxy substituted with a solid support T, benzyl, substituted benzyl, benzyl substituted with a polymer, preferably benzyl substituted with a solid support T, SCH₂.CH₂.CO₂T wherein T is a polymer and preferably SCH₂.CH₂.CO₂T wherein T is a solid support;

Preferred groups Z include

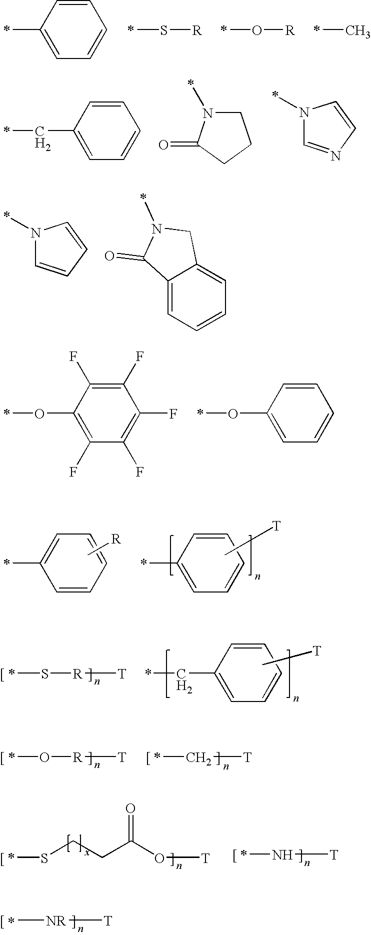

wherein T is a solid support selected from an organic compound, an inorganic compound or magnetised beads. Organic solid supports include, but are not limited to, conventional cross-linked polymers, such as Wang or Merrifield resins, celluloses, cross-linked polyolefins. Inorganic supports include, but are not limited to, silica, and alumina. n is an integer of at least 1, preferably up to 20, 15, 10, 8 or 6. Most preferably n=1, 2, 3, 4, 5 or 6.

Particularly preferred groups Z include

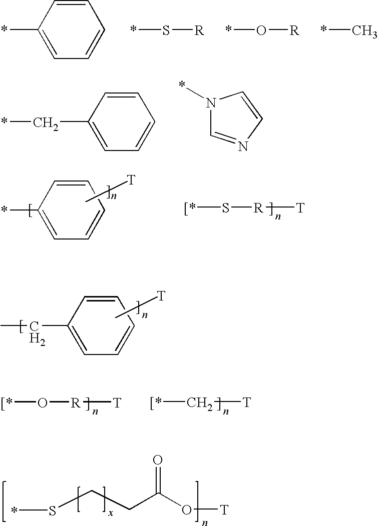

Preferred groups R include

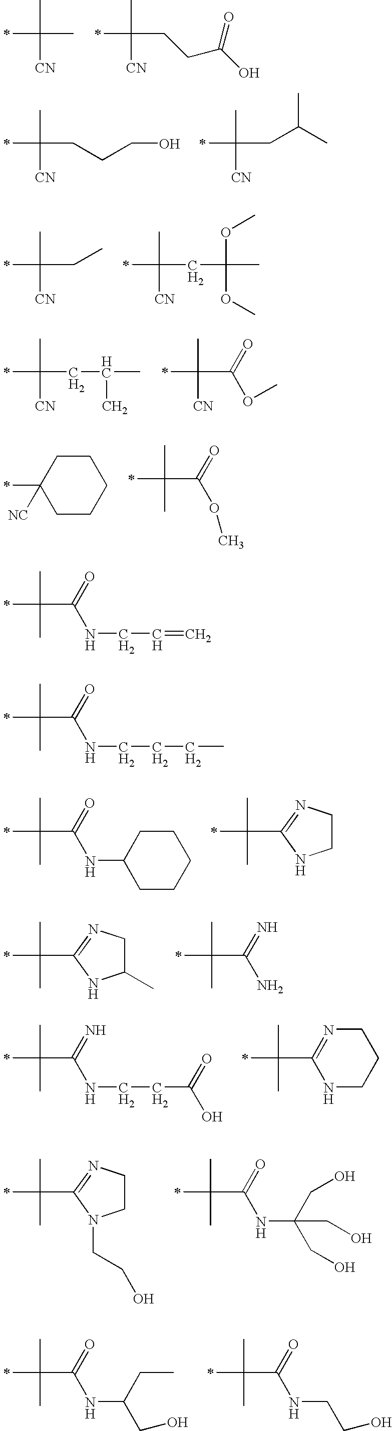

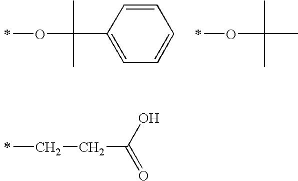

While not being bound by any one mechanism, RAFT and MADIX polymerizations with a singly-functional chain transfer agent (CTA), such as a thiocarbonyl thio, are thought to occur by the mechanism illustrated in Scheme 2. Briefly, an initiator produces a free radical, which subsequently reacts with a polymerizable monomer. The monomer radical reacts with other monomers and propagates to form a chain, Pm., which can react with a CTA. The CTA can fragment, either forming R., which will react with another monomer that will form a new chain, Pn., or Pm., which will continue to propagate. In theory, propagation of Pm. and Pn. will continue until no monomer is left and a termination step occurs. After the first polymerization has finished, in particular circumstances, a second monomer can be added to the system to form a block copolymer. The present invention can also be used to synthesize multiblock, graft, star, gradient, and end-functional polymers.

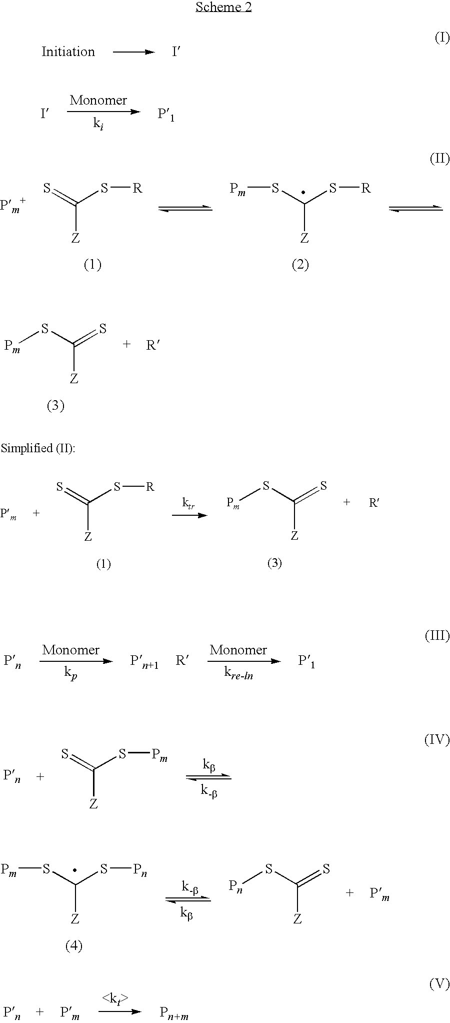

Suitable polymerizable monomers and comonomers of the present invention include methyl methacrylate, ethyl acrylate, propyl methacrylate (all isomers), butyl methacrylate (all isomers), 2-ethylhexyl methacrylate, isobornyl methacrylate, methacrylic acid, benzyl methacrylate, phenyl methacrylate, methacrylonitrile, alpha-methylstyrene, methyl acrylate, ethyl acrylate, propyl acrylate (all isomers), butyl acrylate (all isomers), 2-ethylhexyl acrylate, isobornyl acrylate, acrylic acid, benzyl acrylate, phenyl acrylate, acrylonitrile, styrene, acrylates and styrenes selected from glycidyl methacrylate, 2-hydroxyethyl methacrylate, hydroxypropyl methacrylate (all isomers), hydroxybutyl methacrylate (all isomers), N,N-dimethylaminoethyl methacrylate, N,N-diethylaminoethyl methacrylate, triethyleneglycol methacrylate, itaconic anhydride, itaconic acid, glycidyl acrylate, 2-hydroxyethyl acrylate, hydroxypropyl acrylate (all isomers), hydroxybutyl acrylate (all isomers), N,N-dimethylaminoethyl acrylate, N,N-diethylaminoethyl acrylate, triethyleneglycol acrylate, methacrylamide, N-methylacrylamide, N,N-dimethylacrylamide, N-tert-butylmethacrylamide, N-n-butylmethacrylamide, N-methylolacrylamide, N-ethylolacrylamide, vinyl benzoic acid (all isomers), diethylaminostyrene (all isomers), alpha-methylvinyl benzoic acid (all isomers), diethylamino alpha-methylstyrene (all isomers), p-vinylbenzenesulfonic acid, p-vinylbenzene sulfonic sodium salt, trimethoxysilylpropyl methacrylate, triethoxysilylpropyl methacrylate, tributoxysilylpropyl methacrylate, dimethoxymethylsilylpropyl methacrylate, diethoxymethylsilylpropylmethacrylate, dibutoxymethylsilylpropyl methacrylate, diisopropoxymethylsilylpropyl methacrylate, dimethoxysilylpropyl methacrylate, diethoxysilylpropyl methacrylate, dibutoxysilylpropyl methacrylate, diisopropoxysilylpropyl methacrylate, trimethoxysilylpropyl acrylate, triethoxysilylpropyl acrylate, tributoxysilylpropyl acrylate, dimethoxymethylsilylpropyl acrylate, diethoxymethylsilylpropyl acrylate, dibutoxymethylsilylpropyl acrylate, diisopropoxymethylsilylpropyl acrylate, dimethoxysilylpropyl acrylate, diethoxysilylpropyl acrylate, dibutoxysilylpropyl acrylate, diisopropoxysilylpropyl acrylate, vinyl acetate, vinyl butyrate, vinyl benzoate, vinyl chloride, vinyl fluoride, vinyl bromide, maleic anhydride, N-phenylmaleimide, N-butylmaleimide, N-vinylpyrrolidone, N-vinylcarbazole, butadiene, isoprene, chloroprene, ethylene, propylene, 1,5-hexadienes, 1,4-hexadienes, 1,3-butadienes, and 1,4-pentadienes. Additional suitable polymerizable monomers and comonomers include vinyl acetate, N-vinyl formamide, N-alkylvinylamine, allylamine, N-alkylallylamine, diallylamine, N-alkyldiallylamine, alkylenimine, acrylic acids, alkylacrylates, acrylamides, methacrylic acids, alkylmethacrylates, methacrylamides, N-alkylacrylamides, N-alkylmethacrylamides, styrene, vinylnaphthalene, vinyl pyridine, ethylvinylbenzene, aminostyrene, vinylbiphenyl, vinylanisole, vinylimidazolyl, vinylpyridinyl, dimethylaminomethylstyrene, trimethylammonium ethyl methacrylate, trimethylammonium ethyl acrylate, dimethylamino propylacrylamide, trimethylammonium ethylacrylate, trimethylammonium ethyl methacrylate, trimethylammonium propyl acrylamide, dodecyl acrylate, octadecyl acrylate, and octadecyl methacrylate.

Preferred polymerizable monomers and comonomers include alkylacrylamides, methacrylamides, acrylamides, styrenes, allylamines, allylammonium, diallylamines, diallylammoniums, alkylmethacrylates, alkylacrylates, methacrylates, acrylates, n-vinyl formamide, vinyl ethers, vinyl sulfonate, acrylic acid, sulfobetaines, carboxybetaines, phosphobetaines, and maleic anhydride.

Even more preferred polymerizable monomers and comonomers include alkylmethacrylates, alkylacrylates, methacrylates, acrylates, alkylacrylamides, methacrylamides, acrylamides, and styrenes.

Block copolymers may be made by sequential addition of different monomers to the reaction catalyst. Alternatively statistical polymers may be produced using a mixture of two more different monomers.

The method of the invention may also be used to produce comb star, branched or graft polymers.

The first source of free radicals can be any suitable method of generating free radicals such as thermally induced homolytic scission of a suitable compound(s) (thermal initiators such as peroxides, peroxyesters, or azo compounds), the spontaneous generation from a monomer (e.g., styrene), redox initiating systems, photochemical initiating systems or high energy radiation such as electron beam, X- or gamma-ray radiation. The initiating system is chosen such that under the reaction conditions, there is no substantial adverse interaction of the initiator, the initiating conditions, or the initiating radicals with the transfer agent under the conditions of the procedure. The initiator should also have the requisite solubility in the reaction medium or monomer mixture.

Thermal initiators are chosen to have an appropriate half-life at the temperature of polymerization. These initiators can include one or more of 2,2′-azobis(isobutyronitrile), 4,4′-azobis(4-cyanopentanoic acid, 2-(t-butylazo)-2-cyanopropane, 2,2′-azobis(isobutyramide) dihydrate, 2,2′-azobis(2-methylpropane), 2,2′-Azobis[2-(5-methyl-2-imidazolin-2-yl)propane]dihydrochloride, 2,2′-Azobis[2-(2-imidazolin-2-yl)propane]dihydrochloride, 2,2′-Azobis[2-(2-imidazolin-2-yl)propane disulfate dehydrate, 2,2′-Azobis(2-methylpropionamide)dihydrochloride, 2,2′-Azobis[N-(2-carboxyethyl)-2-methylpropionamidine]tetrahydrate, 2,2′-Azobis[2-(3,4,5,6-tetrahydropyrimidin-2-yl)propane]dihydrochloride, 2,2′-Azobis{2-[1-(2-hydroxyethyl)-2-imidazolin-2-yl]propane}dihydrochloride, 2,2′-Azobis[2-(2-imidazolin-2-yl)propane], 2,2′-Azobis{2-methyl-N-[1,1-bis(hydroxymethyl)-2-hydroxyethyl]propionamide, 2,2′-Azobis{2-methyl-N-[2-(1-hydroxybuthyl)]propionamide}, 2,2′-Azobis[2-methyl-N-(2-hydroxyethyl)propionamide], 2,2′-Azobis(4-methoxy-2,4-dimethyl valeronitrile), 2,2′-Azobis(2,4-dimethyl valeronitrile), Dimethyl 2,2′-azobis(2-methylpropionate), 2,2′-Azobis(2-methylbutyronitrile), 1,1′-Azobis(cyclohexane-1-carbonitrile), 2,2′-Azobis[N-(2-propenyl)-2-methylpropionamide], 1-[(cyano-1-methylethyl)azo]formamide, 2,2′-Azobis(N-butyl-2-methylpropionamide), 2,2′-Azobis(N-cyclohexyl-2-methylpropionamide), t-butyl peroxyacetate, t-butyl peroxybenzoate, t-butyl peroxyoctoate, t-butyl peroxyneodecanoate, t-butylperoxy isobutyrate, t-amyl peroxypivalate, t-butyl peroxypivalate, di-isopropyl peroxydicarbonate, dicyclohexyl peroxydicarbonate, dicumyl peroxide, dibenzoyl peroxide, dilauroyl peroxide, potassium peroxydisulfate, ammonium peroxydisulfate, di-t-butyl, hyponitrite, and dicumyl hyponitrite. Photochemical initiator systems are chosen to have the requisite solubility in the reaction medium or monomer mixture and have an appropriate quantum yield for radical production under the conditions of the polymerization. Examples include benzoin derivatives, benzophenone, acyl phosphine oxides, and photo-redox systems.

Redox initiator systems are chosen to have the requisite solubility in the reaction medium or monomer mixture and have an appropriate rate of radical production under the conditions of the polymerization; these initiating systems can include combinations of oxidants such as potassium peroxydisulfate, hydrogen peroxide, t-butyl hydroperoxide and reductants such as iron(II), titanium(III), potassium thiosulfite, and potassium bisulfite. Other suitable initiating systems are described in recent texts. See, for example, Moad and Solomon, “The Chemistry of Free Radical Polymerization,” Pergamon, London, 1995, pp. 53-95.

Polymerizations of the present invention can occur in any suitable solvent or mixture thereof. Suitable solvents include water, alcohol (e.g., methanol, ethanol, n-propanol, isopropanol, butanol), tetrahydrofuran (THF) dimethyl sulfoxide (DMSO), dimethylformamide (DMF), acetone, acetonitrile, benzene, toluene.

It desirable to choose reaction components (solvent, etc.), such that the components have a low transfer constant towards the propagating radical. Chain transfer to these species will lead to the formation of chains that do not contain an active thiocarbonyl thio group.

In addition to the choice of thiocarbonyl thio, monomer or comonomer, free radical source, and solvent, the choice of polymerization conditions may be also important. The reaction temperature will influence the rate. For example, higher reaction temperatures will typically increase the rate of fragmentation. Conditions may be chosen such that the number of chains formed from initiator-derived radicals is minimized to an extent consistent with obtaining an acceptable rate of polymerization. Termination of polymerization by radical-radical reactions will lead to chains that contain no active group and therefore cannot be reactivated. The rate of radical-radical termination is proportional to the square of the radical concentration. Furthermore, in the synthesis of block, star, or branched polymers, chains formed from initiator-derived radicals may constitute a linear homopolymer impurity in the final product. The reaction conditions for these polymers therefore may require careful choice of initiator concentration and, where appropriate, the rate of initiator feed.

As a general guide in choosing conditions for the synthesis of narrow dispersity polymers, the concentration of initiator(s) and other reaction conditions (solvent(s), temperature, pressure) may be chosen such that the molecular weight of polymer formed in the absence of the CTA is at least twice that formed in its presence. In polymerizations where termination is solely by disproportionation, this may equate to choosing an initiator concentration such that the total moles of initiating radicals formed during the polymerization is less than 0.5 times that of the total moles of CTA. More preferably, conditions may be chosen such that the molecular weight of polymer formed in the absence of the CTA is at least 5-fold that formed in its presence.

The polydispersity of polymers and copolymers synthesized by the method of the present invention may be controlled by varying the ratio of the numbers of molecules of CTA to initiator. A lower polydispersity is obtained when the ratio of CTA to initiator is increased. Conversely, a higher polydispersity is obtained when the ratio of CTA to initiator is decreased. Preferably, conditions are selected such that polymers and copolymers have a polydispersity less than about 1.5, more preferably less than about 1.3, even more preferably less than about 1.2, and yet more preferably less than about 1.1. In conventional free radical polymerizations, polydispersities of the polymers formed are typically in the range of 1.6-2.0 for low conversions (<10%) and are substantially greater than this for higher conversions

With these provisos, the polymerization process according to the present invention may be performed under the conditions typical of conventional free-radical polymerization. Polymerizations employing the above described thiocarbonyl thio compounds are suitably carried out at temperatures in the range −20 to 200° C., preferably 20 to 150° C., more preferably 50 to 120° C., or even more preferably 60 to 90° C.

In the case of emulsion or suspension polymerization the medium may be predominately water and the conventional stabilizers, dispersants and other additives can be present. For solution polymerization, the reaction medium may be chosen from a wide range of media to suit the monomer (s) used.

The use of feed polymerization conditions allows the use of chain transfer agents with lower transfer constants and allows the synthesis of block polymers that are not readily achieved using batch polymerization processes. If the polymerization is carried out as a feed system the reaction can be carried out as follows. The reactor is charged with the chosen medium, the chain transfer agent and optionally a portion of the monomer(s). The remaining monomer(s) is placed into a separate vessel. Initiator is dissolved or suspended in the reaction medium in another separate vessel. The medium in the reactor is heated and stirred while the monomer+medium and initiator+medium are introduced over time, for example by a syringe pump or other pumping device. The rate and duration of feed is determined largely by the quantity of solution the desired monomer/chain transfer agent/initiator ratio and the rate of the polymerization. When the feed is complete, heating can be continued for an additional period.

The inventors have unexpectedly found that reacting a supported thiocarbonyl thio compound (chain transfer agent) with the non-supported equivalent (i.e. with the identical R′ groups, but different Z groups) allows the polymerisation to be better controlled. Hence a further aspect of the invention provides: a method according to the invention comprising the step of reacting a first supported thiocarbonyl thio compound of Formula (3) or Formula (4) with the olefinically unsaturated monomer (Q) and the first source of free radical to form a polymer of Formula (6) or Formula (7) in the presence of a second non-supported thiocarbonyl compound and the first and second thiocarbonyl having identical groups R′.

This may also apply to any RAFT process. Hence a further aspect of the invention provides: a method of carrying out a reversible-addition-fragmentation chain transfer (RAFT) polymerisation comprising the steps of reacting olefinically unsaturated monomers with a first supported chain transfer agent, in the presence of a second unsupported chain transfer agent, in the presence of a free radical source, to form a polymer.

By supported we mean that the chain transfer agent is attached to a solid support or polymer, such as those discussed above. However, the rest of the chain transfer agent is identical. The chain transfer agents used may be any known in the art, such as the thiocarbonyl compounds shown in WO 98/01478 or WO 02/26836. They may be attached to supports or polymers by the techniques discussed herein.

The non-supported chain transfer agent is preferably in solution. Preferably more supported than non-supported agent is used.

The invention has wide applicability in the field of free radical polymerization and can be used to produce polymers and compositions for coatings, including clear coats and base coat finishes for paints for automobiles and other vehicles or maintenance finished for a wide variety of substrates. Such coatings can further include pigments, durability agents, corrosion and oxidation inhibitors, rheology control agents, metallic flakes and other additives. Block and star, and branched polymers can be used as compatibilisers, thermoplastic elastomers, dispersing agents or rheology control agents. Additional applications for polymers of the invention are in the fields of imaging, electronics (e.g., photoresists), engineering plastics, adhesives, sealants, and polymers in general.

The invention also provides supported compounds for use in the method of the invention comprising the formula:

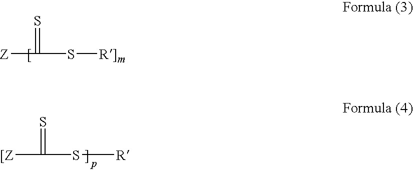

### Where:

Z is a solid support or a solid support attached via a linker to the thiocarbonyl thio moiety,
m=an integer of at least 1,
p=an integer of at least 1,
R′ is selected from the group consisting of alkyl, substituted alkyl, alkoxy, substituted alkoxy, an aromatic saturated or unsaturated carbocyclic or heterocyclic ring, optionally substituted with one or more substituents, amino alkyl, cyanoalkyl, hydroxylalkyl, saturated and unsaturated amido; an organometallic species, a polymer chain and any of the foregoing substituted with one or more CN or OH groups.

Polymers attached to the supported thiocarboxylthio compounds are provided having the formula:

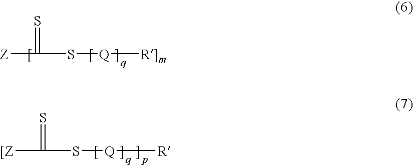

### Where:

Z is a solid support or a solid support attached via a linker to the thiocarboxylthio moiety,
m=an integer of at least 1,
p=an integer of at least 1,
q=an integer of at least 2,
R′ is selected from the group consisting of alkyl, substituted alkyl, alkoxy, substituted alkoxy, an aromatic saturated or unsaturated carbocyclic or heterocyclic ring, optionally substituted with one or more substituents, amino alkyl, cyanoalkyl, hydroxylalkyl, saturated and unsaturated amido; an organometallic species, a polymer chain and any of the foregoing substituted with one or more CN or OH groups,
Q is at least one olefinically unsaturated monomer, optionally two or more different olefinically unsaturated monomers.

Preferably Z is selected from

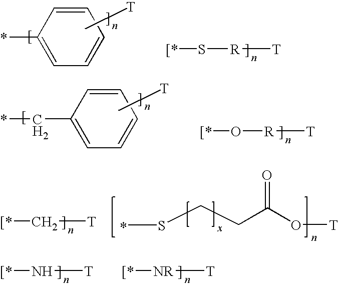

wherein T is a solid support selected from an organic compound, an inorganic compound or magnetised beads,
R is selected from a group consisting of alkyl, substituted alkyl, alkoxy, substituted alkoxy, an aromatic saturated or unsaturated carbocyclic or heterocyclic ring, optionally substituted with one or more substituents, amino alkyl, cyanoalkyl, hydroxylalkyl, saturated and unsaturated amido; an organometallic species, a polymer chain and any of the foregoing substituted with one or more CN or OH groups,
n=an integer of at least 1, preferably up to 20, 15, 10, 8 or 6.
Most preferably n=1, 2, 3, 4, 5 or 6.

Preferably

Z, m, p, q, n, R′, R and Q are as defined above for the method of the invention.

Polymers obtained or obtainable by a method of the invention are also provided.

Scheme 3 illustrates a process using a mono-chain transfer agent, that is wherein m or p=1 in Formulae (3) or (4). In this process i, j=1, 2.

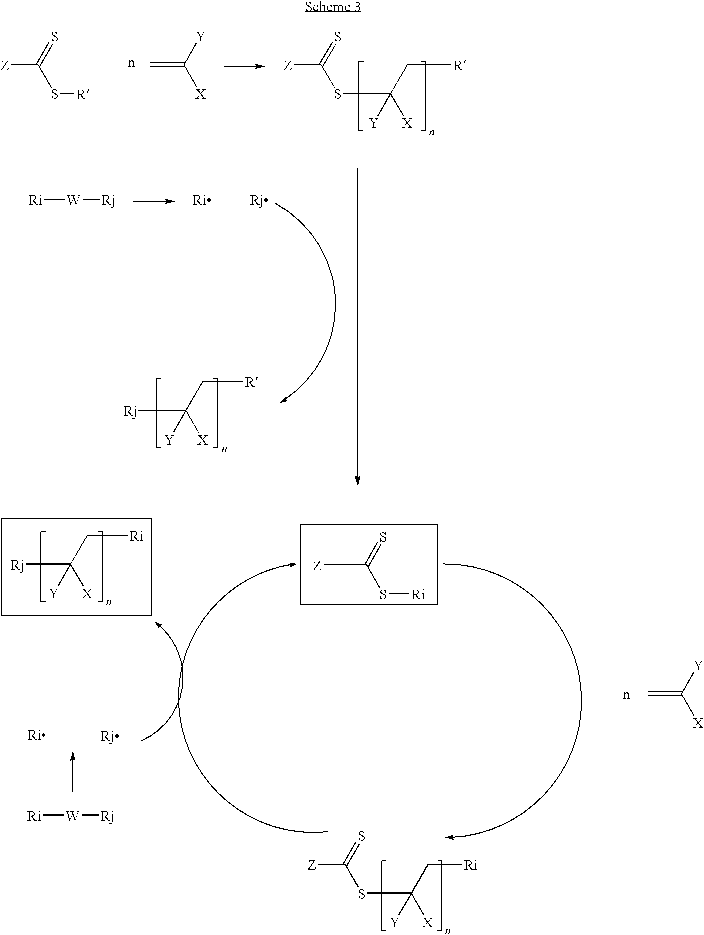

Scheme 4 illustrates an alternative process wherein R is multifunctional. R may be a star-compound or may be a cross-linked polymer bead or other support. In this process i, j=1, 2.

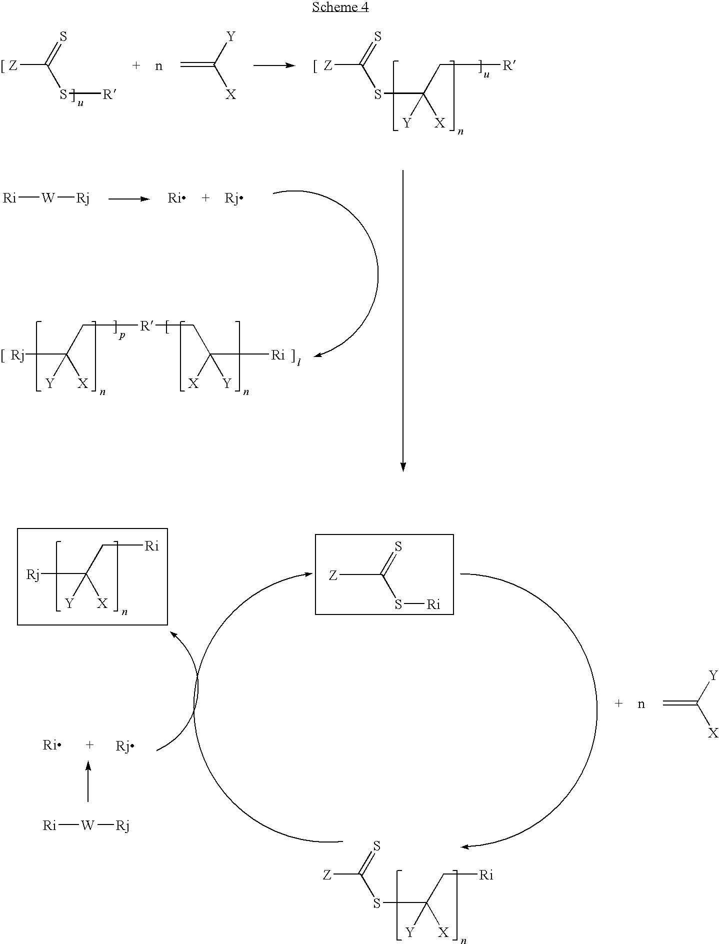

Scheme 5 shows a process wherein Z is difunctional and R₁ and R₂ may be different.

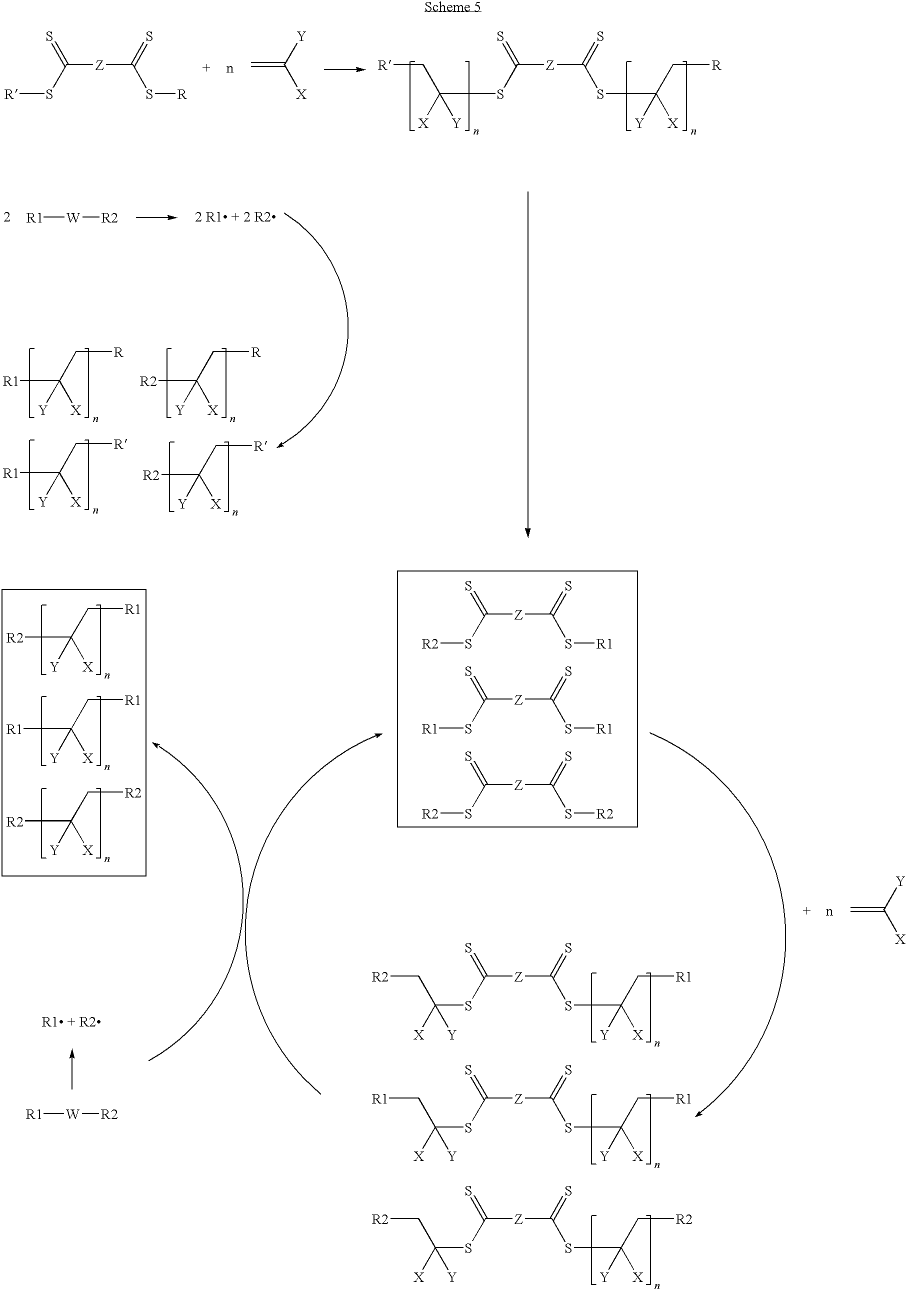

Scheme 6 illustrates use of a multifunctional group Z. Rx may be R₁ or R₂.

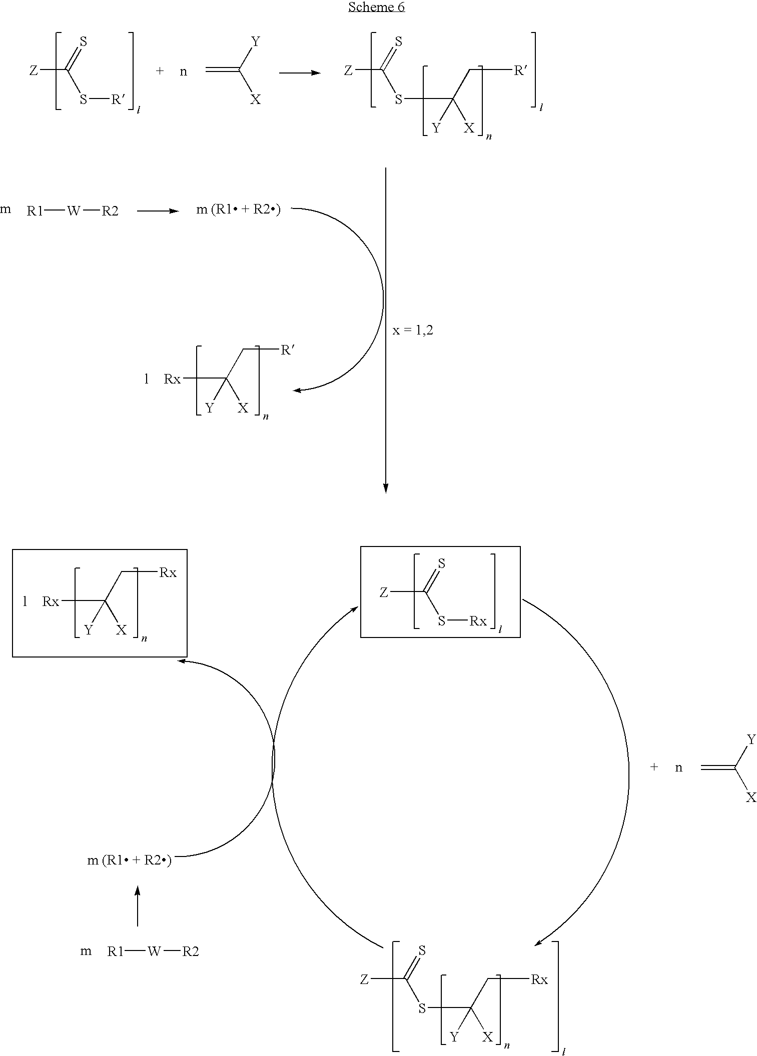

Scheme 7 illustrates use of a supported chain transfer agent.

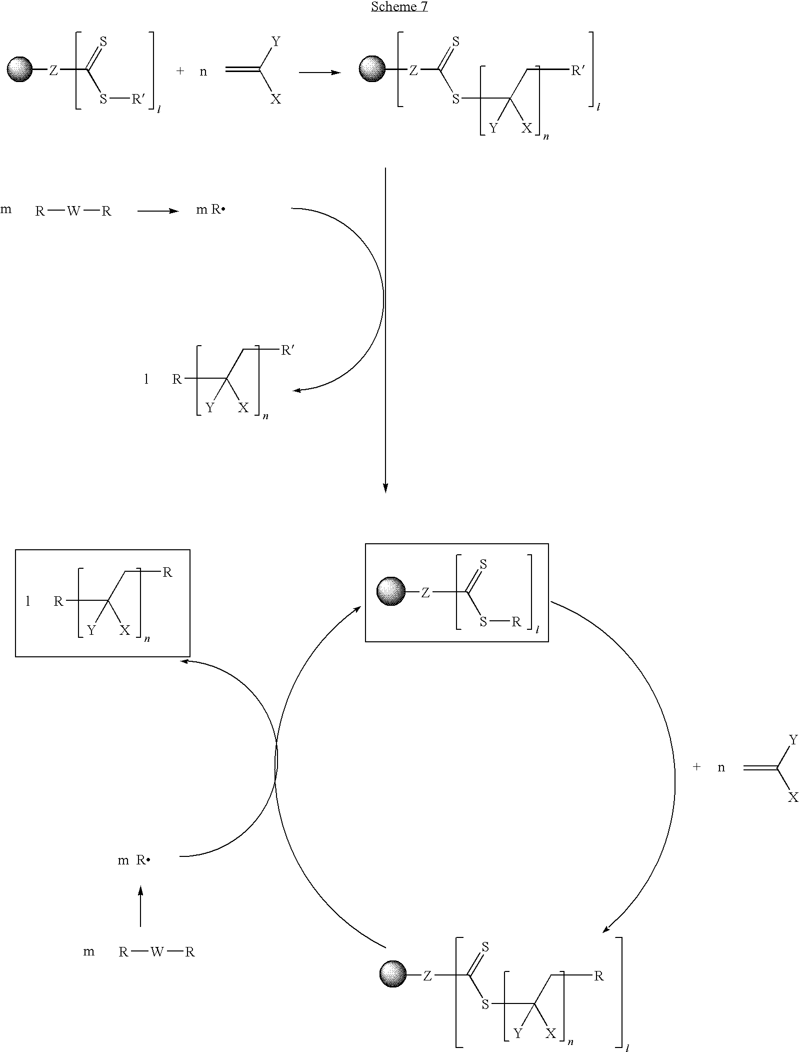

The invention is further described by means of example but not in any limitative sense.

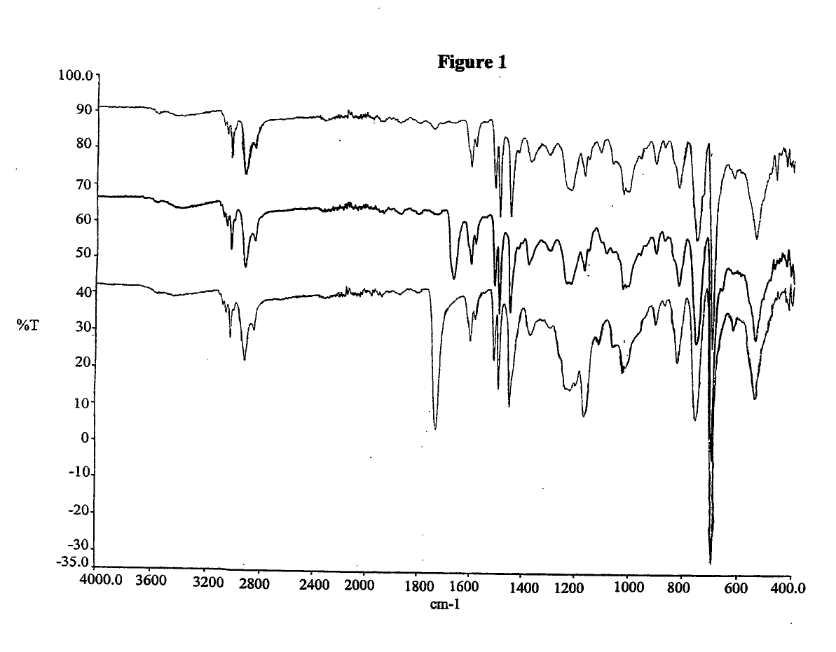

FIG. 1 shows FTIR for (top) Wang resin, (middle) Wang—ICSPE and (bottom) Wang poly(methyl acrylate) (PMA)—ICSPE made according to the examples.

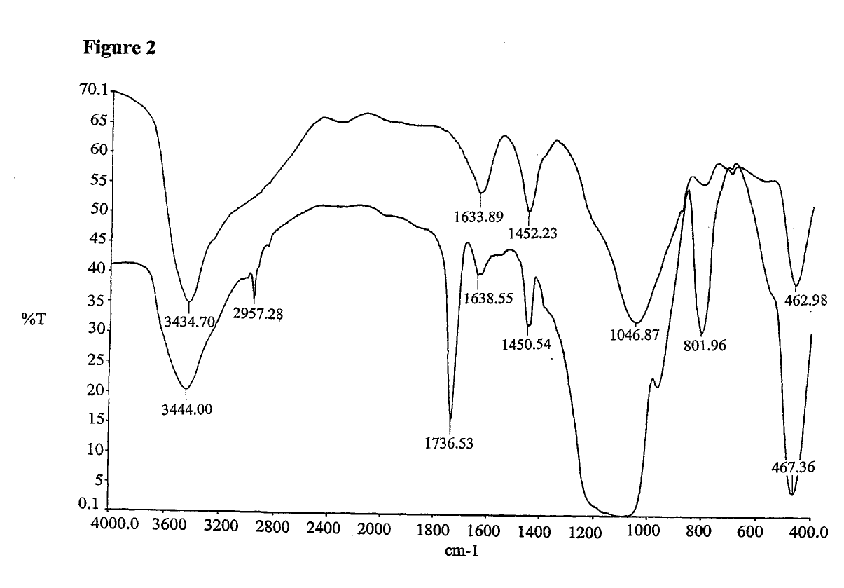

FIG. 2 shows FTIR of Silica supported CTA (top) and polymethacrylate on silica—CTA (bottom) Band at 1736 cm⁻¹ corresponds to C═O of polymethacrylate.

In each of the following examples the following were observed:
UV-V is of the recovered chain transfer agent (CTA) showed similar λₘₐₓ as the original CTA.
GC-MS confirmed the nature of the recovered CTA.
¹H-NMR of the recovered polymer showed the disappearance of the characteristic peaks of the dithioester moiety at 7.94 ppm.
End group analysis of the polymer via pyrolysis GC-MS showed the absence of dithioester moiety.

### Example 1

### Synthesis of Compound 1, Table 1

A solution of methyl methacrylate (MMA, 12.200 g, 121.8 mmol), S-methoxycarbonylphenylmethyl dithiobenzoate (MCPDB, 0.074 g, 0.244 mmol), and α,α′-azoisobutyronitrile (AIBN; 0.004 g, 0.024 mmol) was placed in an ampoule and degassed by flowing nitrogen gas through the solution for 5 min. The ampoule was placed in a water bath pre-heated to 60° C. and samples were taken out at various times to monitor monomer conversion. Each sample was placed in an ice bath to quench the reaction. The percentage conversions were measured by ¹H-NMR and molecular weights and PDI were analyzed by SEC. Upon completion of the reaction, the polymer was precipitated in cold hexane and recovered by filtration.

In a second step, the poly(methyl methacrylate) formed (Mₙ=29,709 g/mol, 0.161 g, 5.42×10⁻⁶ mol) and α,α′-azoisobutyronitrile (AIBN, 164.12 g/mol, 0.0179 g, 10.84×10⁻⁵ mol) were added in an ampoule in 5 mL of toluene. Nitrogen gas was then flowed through the solution for 5 min. The ampoule was placed in an oil bath pre-heated to 80° C. The sample was left for 2.5 hrs and placed into an ice bath to quench the reaction. The sample was reprecipitated in cold hexane and then filtered. The precipitated polymer was dried in a vacuum oven overnight. The polymer was characterised by ¹H-NMR, UV-Vis, GPC and pyrolysis GC-MS. The recovered CTA was obtained by removal in-vacuo of the filtrate's solvent and analysed by GC-MS and UV-Vis.

### Example 2

### Synthesis of Polymer with End Groups Similar to Compound 1, Table 1 (Reactions with α,α′-azoisobutyronitrile, AIBN)

A solution of monomer, chain transfer agent (CTA) (0.244 mmol), and α,α′-azoisobutyronitrile (0.024 mmol) was placed in an ampoule and degassed by flowing nitrogen gas through the solution for 5 min. The ampoule was placed in a water bath pre-heated to 60° C. and samples were taken out at various times to monitor monomer conversion. Each sample was placed in an ice bath to quench the reaction. The percentage conversions were measured by ¹H-NMR and molecular weights and PDI were analyzed by SEC. Upon completion of the reaction, the polymer was precipitated in cold hexane and recovered by filtration.

The polymer synthesised above was weighed in the range of 0.3-1.0 g (Mₙ between 5,000 and 40,000 g mol⁻¹) in an ampoule. AIBN was added in the ampoule with 5 mL of toluene (various molar ratios were tested). Nitrogen gas was then flowed through the solution for 5 min. The ampoules were placed in an oil bath pre-heated to 80° C. The sample was left for 2.5 hrs and placed into an ice bath to quench the reaction. The sample was reprecipitated in cold hexane and then filtered. The solvent in the filtrate was removed in vacuo and the resulting solid was analysed with GC-MS and UV-Vis. The precipitated polymer was dried in a vacuum oven for an overnight. The polymer was characterised by ¹H-NMR, UV-Vis, GPC and pyrolysis GC-MS.

### Example 3

### Synthesis of Polymer with End Groups Similar to Compound 7, Table 1 (Reaction with α,α′-azobis(cyclohexanecarbonitrile), ACHN)

A solution of monomer, chain transfer agent (CTA) (0.244 mmol), and α,α′-azoisobutyronitrile (0.024 mmol) was placed in an ampoule and degassed by flowing nitrogen gas through the solution for 5 min. The ampoule was placed in a water bath pre-heated to 60° C. and samples were taken out at various times to monitor monomer conversion. Each sample was placed in an ice bath to quench the reaction. The percentage conversions were measured by ¹H-NMR and molecular weights and PDI were analyzed by SEC. Upon completion of the reaction, the polymer was precipitated in cold hexane and recovered by filtration.

The polymer synthesised above was weighed in the range of 0.3-1.0 g (Mₙ between 5,000 and 40,000 g mol⁻¹) in an ampoule. ACHN was added in the ampoule with 5 mL of toluene (various molar ratios were tested). Nitrogen gas was then flowed through the solution for 5 min. The ampoules were placed in an oil bath pre-heated to 100° C. The sample was left for 2.5 hrs and placed into an ice bath to quench the reaction. The sample was reprecipitated in cold hexane and then filtered. The solvent in the filtrate was removed in vacuo and the resulting solid was analysed with GC-MS and UV-Vis. The precipitated polymer was dried in a vacuum oven for an overnight. The polymer was characterised by ¹H-NMR, UV-Vis, GPC and pyrolysis GC-MS.

### Example 4

### Synthesis of Polymer with End Groups Similar to Compound 8, Table 1 (Reaction with Dicumyl Peroxide)

A solution of monomer, chain transfer agent (CTA) (0.244 mmol), and a azoisobutyronitrile (0.024 mmol) was placed in an ampoule and degassed by flowing nitrogen gas through the solution for 5 min. The ampoule was placed in a water bath pre-heated to 60° C. and samples were taken out at various times to monitor monomer conversion. Each sample was placed in an ice bath to quench the reaction. The percentage conversions were measured by ¹H-NMR and molecular weights and PDI were analyzed by SEC. Upon completion of the reaction, the polymer was precipitated in cold hexane and recovered by filtration.

The polymer synthesised above was weighed (0.5 g) in an ampoule with dicumyl peroxide (in the ratio of 20 molar equivalents) and 5 mL of xylene. The solution was degassed for 5 min by nitrogen bubbling. The ampoule was then placed in an oil bath pre-heated to 130° C. After 2 hrs, the ampoule was removed and placed into an ice bath to quench the reaction. The sample was dissolved in dichloromethane, precipitated in cold hexane and filtered. The product was analysed with ¹H-NMR, UV-Vis, and pyrolysis GC-MS.

### Example 5

### Synthesis of Wang Resin CTA

Wang resin beads, 4.468 g (8.13 mmol, 1.82 mmol g⁻¹ OH functionality), were placed in a 250 mL round bottom flask, equipped with a magnetic stirrer and placed in an oil bath. Dry tetrahydrofuran (100 mL) was added to the flask and the suspension was stirred at low speed. Carbon disulphide, 10 mL (0.132 mol) was added to the flask and further stirred for 0.5 h at ambient temperature before increasing the temperature to reflux for 6 h. After reaction, THF and excess of carbon disulphide were removed in vacuo and tetrahydrofuran (100 mL) was further added to the flask. The suspension was stirred at low speed under dry condition with 10.0 mmol triethylamine. Methyl-α-bromophenylacetate (10.0 mmol) was further added dropwise to the flash. The reaction temperature was increased to reflux and left overnight. The resin was washed with water (to remove the quanternary ammonium salt of triethylamine), THF and dichloromethane (to remove non-attached impurities). The resin was dried in vacuo and analysed by FTIR.

### Example 6

### Support Based on Wang Resin

### Synthesis of Wang Chain Transfer Agent (Wang-ICSPE)

Wang Resin, 4.00 g (7.28 mmol, 1.82 mmol g⁻¹ OH functionality), was placed in a 250 mL round bottom flask, equipped with a magnetic stirrer and placed in an oil bath. Toluene (100 mL) and potassium hydroxide, 0.02 g, (0.36 mmol) were added to the flask under N₂ atmosphere. 2-(Imidazole-1-carbothioylsulfanyl)-propionic acid ethyl ester (ICSPE), 2.50 g, (10.2 mmol) was added to the flask and the reaction temperature was increased to 60° C. for 16 h. The functionalised resin was washed with toluene and THF. The final product was analysed by FTIR, Particle Size Analysis and Colour Matching Analyser. This is shown in FIG. 1.

top is wang
middle is wang-ICSPE
bottom is wang-poly(methyl acrylate) (PMA)-ICSPE

### Example 7

### Support Based on Merrifield Resin

### Synthesis of Merrifield Chain Transfer Agent (CTA): Merrifield-MCPDB

Merrifield Resin (Merrifield-Cl, 65.2 g, 61.2 mmol) was weighed in a round bottom flask with a magnetic stirrer. Elemental sulphur (4.00 g, 125 mmol) was placed into the flask. Tetrahydrofuran (500 mL) was then added and the suspension was stirred gently. Sodium methoxide (27.0 mL, 125 mmol) was transferred into the flask dropwise. The reaction was left overnight. The solid was washed with warm toluene (250 mL×3), warm THF (200 mL×2), warm H₂O (100 mL×3), then a mixture of water and THF (1:1) (100 mL×2), warm THF (50×2) and warm toluene (100 mL×2). Tetrahydrofuran (30 mL) was added in to the dried solid and alpha bromophenyl methyl ester (18.88 g, 79.94 mmol) was placed into the suspension. The suspension was refluxed for 6 hrs. The solid was filtered and wash with toluene (200 mL×3), warm THF (200×2), then a mixture of water and THF (1:1) (100 mL×2), warm THF (200 mL×2), dichloromethane (150×1), warm THF (200×2) and warm toluene (200 mL×2). The product was dried and analysed with FTIR, Raman and elemental analysis. The product (orange red) was dried in vacuo and analysed with FTIR, Raman and elemental analysis.

FTIR of Mer-MCPDB (cm⁻¹); 1720 C═O; 1605, 1493, 1451 aromatic skeleton; 1277, C—O

FTIR of Mer-PMA-MCPDB (cm^{-1);} 1738 C═O of PMA

EA. % S=2.05%

The following table lists further polymerizations using non-attached polymers.

TABLE 1

| | Final product | Intermediate Polymer |
|---|---|---|
| 1 | 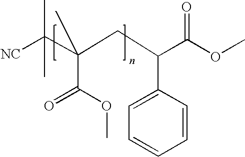 | 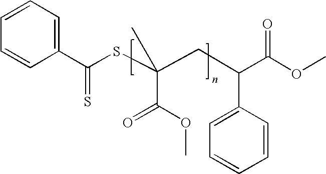 |
| 2 | 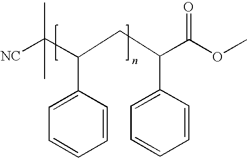 | 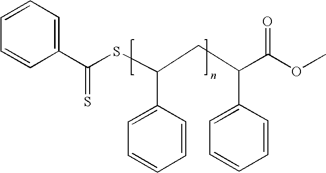 |
| 3 | 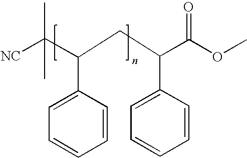 | 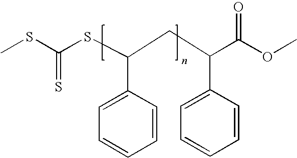 |
| 4 | 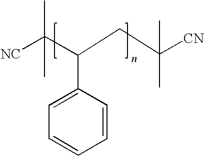 | 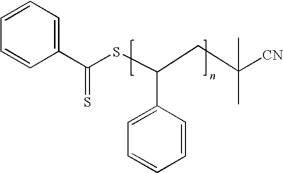 |
| 5 | 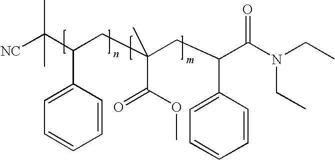 | 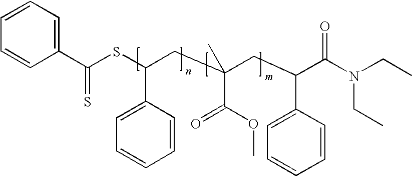 |
| 6 | 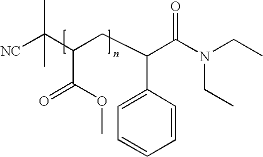 | 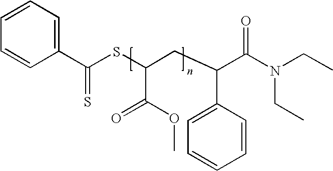 |
| 7 | 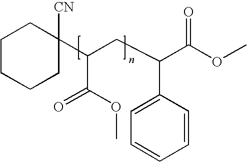 | 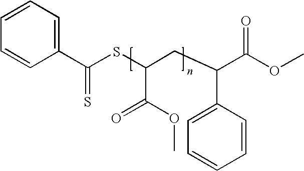 |
| 8 | 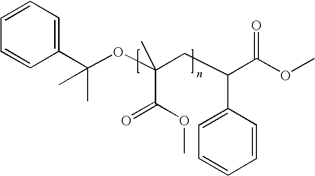 | 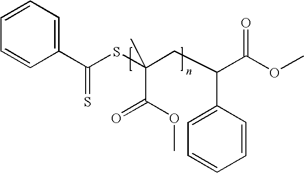 |
| 9 | 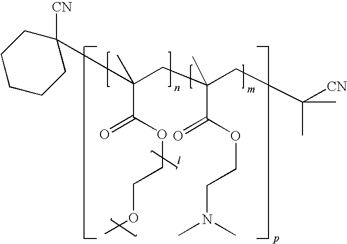 | 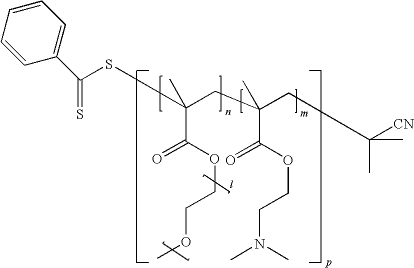 |
| 10 | 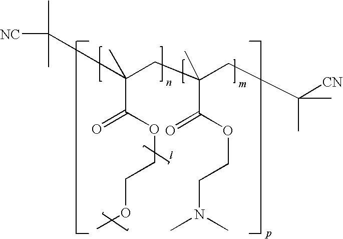 | 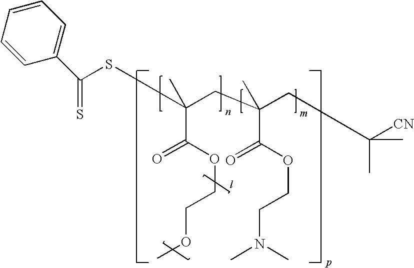 |
| 11 | 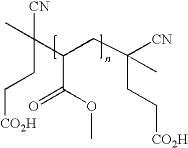 | 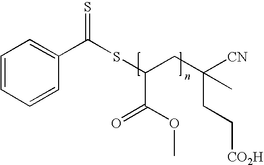 |

| | Radical source/ | Recovered |
|---|---|---|
| | Reaction conditions | Raft agents |
| 1 | 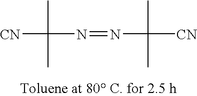 | 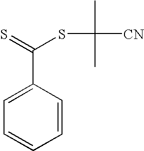 |
| 2 | 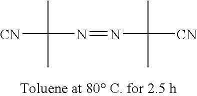 | 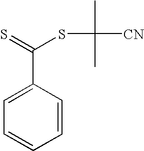 |
| 3 | 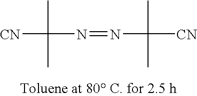 | 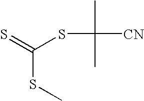 |
| 4 | 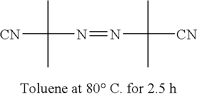 | 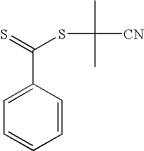 |
| 5 | 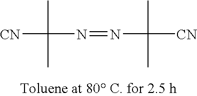 | 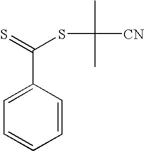 |
| 6 | 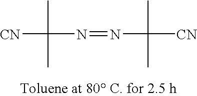 | 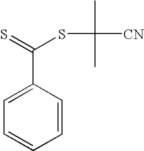 |
| 7 | 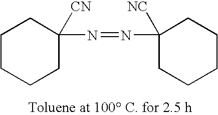 | 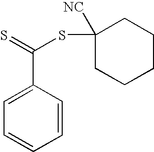 |
| 8 | 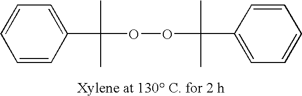 | 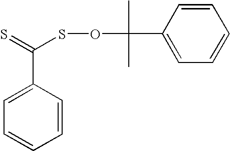 |
| 9 | 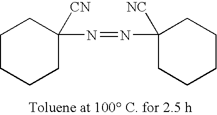 | 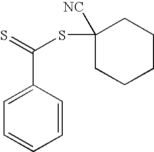 |
| 10 | 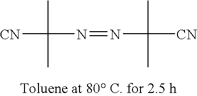 | 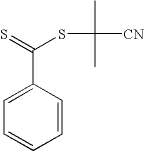 |
| 11 | 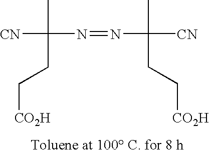 | 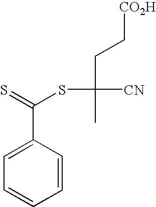 |

### Example 8

### Polymerisation of Methyl Acrylate (MA) from the Wang and Merrifield Resins

A solution of methyl acrylate, chain transfer agent (CTA) (0.244 mmol), and α,α′-azoisobutyronitrile (0.024 mmol) was placed in an ampoule and degassed by flowing nitrogen gas through the solution for 5 min. The ampoule was placed in a water bath pre-heated to 60° C. After a fixed time, the suspension was filtered to separate the polymer attached to the resin from the solution.

### Example 9

### Polymer/Resin CTA Recovery

A sample of example 7 (0.3 g) was placed in a reaction ampoule. AIBN (20 molar equivalents) and toluene (5 mL) were added in the ampoule. The solution was purged by Nitrogen bubbling for 5 mins. The solution was heated at 80° C. for 2.5 h. The suspension was then filtered to separate the resin from the solution. The solvent of the solution was removed in vacuo and the resulting solid was analysed by size exclusion chromatography (SEC). The resin was dried in a vacuum oven and analysed by SEM, particle size analyser and FTIR.

ATR FTIR of the Resin after Polymerisation Showed Absorptions at 1733 cm⁻¹ and 1714 cm⁻¹ Characteristic of the Carbonyl of the PMA.

Scanning electron microscopy showed that the spherical shape of the bead as retained after modification and further polymerization. The resin size, however, increases when first modified, and increases further after polymerization. After reaction with AIBN in toluene, the beads regain the size of the modified resin.

Particle size analysis (PSA) confirmed this observation. In the case of Wang resin, the size of the original beads, modified beads, polymerized beads and recovered beads were 80.10, 88.47, 113.4 and 90.01 μm, respectively. In the case of the Merrifield resin, the average particle sizes were 79.24, 98.47, 134.7 and 103.0 μm, respectively.

### Example 10

### Polymerisation of Methyl Acrylate (MA) Using Merrifield-MCPDB

The polymerizations were processed in the ratios of 250:1:0.1 of monomer:CTA:AIBN, respectively. Resin (5.00 g) and AIBN were added to a Schlenk tube contained with 5 mL of toluene and monomer. The mixtures were stirred gently for 5 min before flushing with nitrogen gas. The reaction was left for 24 hr. The resin was then washed with warn THF (20 mL×3), DCM (50 mL×2), toluene (50 mL×1), warm THF (20×2) (or by Soxhlet extraction using THF for 5 hours. The first and last wash solvents were kept, and analysed with GPC to confirm that no free polymeric chain was left. The resin was then cleaved by 20 equivalents of AIBN in 5 mL of toluene. The solvent was removed and the sample was analysed with GPC. (M_{n non controlled}=256 000, PDI=1.44; M_{n controlled}=13 950, PDI=1.24)

The second cycle polymerisation was processed in the ratios of 250:1:0.1 of monomer:CTA:AIBN, respectively. Resin (2.5 g) and AIBN were added to a Schlenk tube contained with 2.5 mL of toluene and monomer. The mixtures were stirred gently for 5 min before flushing with nitrogen gas. The reaction was left for 24 hr. The washing process was followed as in the first cycle. (M_{n controlled}=14600, PDI=1.17)

### Example 11

### Polymerisation of Methyl Acrylate (MA) Using Merrifield-MCPDB+Free MCPDB

A similar procedure as above was followed, using a ratio MA (5.1654 g): Merrifield-MCPDB (0.75 g. 0.24 mmols): MCPDB (0.0726 g, 0.024 mmols):AIBN (0.004 g, 0.0024 mmols) ratio=250:1:1:0.1. The mixture was added to toluene (100% w/w of MA) in a 100 mL round bottom flask, and N₂ gas was flushed through the flask for 10 mins. The reaction was left at 60 C for 49 hours.

### SEC; Free Polymer, Mn=7410, PDI=1.20

### Cleaved PMA-CN, Mn=8717, PDI=1.11

The resins were characterized by their swelling factor and colour matching determination.

### Swelling Factors

| | Solvent | | |
|---|---|---|---|
| Resin Type | Tetrahydrofuran | Toluene | Water |
| Merrifield-Cl (0.74 mmol Cl/g) | 6.98 | 6.91 | 1.00 |
| 100-200 mesh | | | |
| Merrifield-MCPDB (0.32 mmol/g) | 4.95 | 4.13 | 1.00 |
| Merrifield-Cl (3.58 mmol/g) | 6.65 | 6.27 | 1.00 |
| 300-500 mesh | | | |
| Merrifield-MCPDB (1.58 mmol/g) | 4.40 | 3.50 | 1.00 |
| Wang (1.82 mmol/g) | 6.84 | 4.72 | 1.00 |
| Wang-MCPDB (0.36 mmol/g) | 3.28 | 3.08 | 1.00 |

### Colour Matching Determination

SPIN D65/10°

| | Parameters | | | | |
|---|---|---|---|---|---|
| Resin Type | a* | b* | C* | L* | h° |
| Merrifield-Cl (3.58 mmol/g) | −0.78 | 4.01 | 4.08 | 94.14 | 100.96 |
| Merrifield-MCPDB (0.32 mmol/g) | 31.41 | 25.61 | 40.52 | 39.19 | 61.17 |

### Example 12

### Inorganic Supported Chain Transfer Agents (CTA's)

Silica supported S-methoxycarboyl-α-phenylmethyl dithiobenzoate was prepared by reacting a derivatisable silicate linker, chloromethylphenyltrimethoxysilane, with silica, activated by refluxing in hydrochloric acid, to give chloromethylphenyl derivatised silica. This was subsequently converted to the sodium dithiobenzoate derivatised silica by reacting with elemental sulphur and sodium methoxide. The CTA was finally made by reacting the sodium dithiobenzoate derivatised silica with, but not limited to, methyl abromophenyl acetate. The loading of the CTA on the silica was determined from the sulphur content in the final product using elemental analysis.

Silica supported S-methoxycarboyl-α-phenylmethyl propanetrithiocarbonate was prepared by reacting a derivatisable silicate linker, (3-mercaptopropyl)trimethoxysilane, with silica, activated by refluxing in hydrochloric acid, to give silica supported propanethiol. This was subsequently converted to the sodium propanetrithiocarbonate derivatised silica by reacting with potassium hydroxide and carbon disulphide. The CTA was finally made by reacting the sodium propanetrithiocarbonate derivatised silica with, but not limited to, methyl α-bromophenyl acetate. The loading of the CTA on the silica was determined from the sulphur content in the final product using elemental analysis.

### Example 13

### Polymerisation of an Unsaturated Molecule Using an Inorganic Supported CTA

The CTA was suspended in a solution of methyl acrylate in toluene. To the suspension was added AIBN, with a ratio of 500:1:0.1 (monomer:CTA:initiator), degassed with nitrogen for 10 mins and heated to 60° C. for 24 h. The solution was cooled, filtered and the silica washed with tetrahydrofuran. The filtrate was analysed by gel permeation chromatography (GPC). The silica was washed with toluene and tetrahydrofuran until no free polymer was present on the silica.

### Example 14

### Polymerisation of an Unsaturated Molecule Using an Inorganic Supported CTA with an Additive

The CTA was suspended in a solution of methyl acrylate in toluene. To the suspension was added AIBN and S-methoxycarboyl-α-phenylmethyl dithiobenzoate (MCPDB), with a ratio of 500:1:0.5:0.1 (monomer:inorganic supported CTA:free CTA:initiator), degassed with nitrogen for 10 mins and heated to 60° C. for 24 h. The solution was cooled, filtered and the silica washed with tetrahydrofuran. The filtrate was analysed by gel permeation chromatography (GPC). The silica was washed with toluene and tetrahydrofuran until no free polymer of free CTA was present on the silica.

### Example 15

### Cleavage of Polymer from the Inorganic Supported CTA

The inorganic supported polymer was suspended in toluene. To the suspension was added AIBN, with a ratio of 10:1 (AIBN:inorganic supported CTA), degassed for 10 mins and heated to 60° C. for 2 h. The solution was cooled, filtered and the silica washed with tetrahydrofuran. The filtrate was analysed by gel permeation chromatography (GPC).

### Example 16

### Activation of Silica

Silica (25 g) was suspended in water (100 cm³). To the suspension was added Conc. HCl (20 cm³, 37% sol.) and heated to 90° C. for 5 h. The solution was cooled and the silica filtered off, washed with water (1.5 L) and acetone (0.5 L). The silica was then dried under vacuum at 50° C.

### Silica Supported Phenylmethylchloride

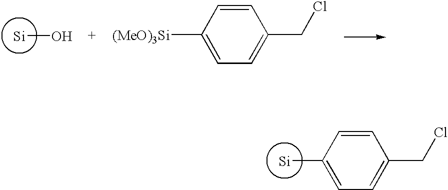

To toluene (25 cm³) was added silica (4 g) and degassed with N₂ for 30 mins. To the slurry was added 4-(chloromethyl)phenyltrimethoxysilane (0.35 g, 1.42 mmol) and heated to 80° C. for 2.5 h. The solution was cooled and the solid filtered off, washed with toluene (200 cm³) and diethyl ether (200 cm³) then dried under vacuum.

### Silica Supported Dithiobenzoate Sodium Salt

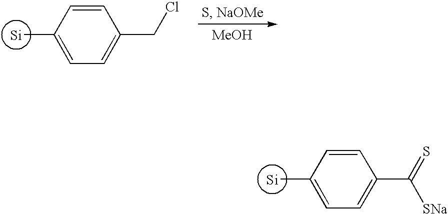

To methanol (30 cm³) was added the benzyl chloride functionalised silica (4 g, 1.42 mmol) sulphur (0.091 g, 2.84 mmol), NaOMe (0.569 g, 25% in methanol) and heated to 70° C. and left to stir overnight. The solution was cooled and the solid filtered off, washed with methanol (200 cm³) and diethyl ether (200 cm²) then dried to yield a cream solid.

### Silica supported S-methoxycarboyl-α-phenylmethyl dithiobenzoate

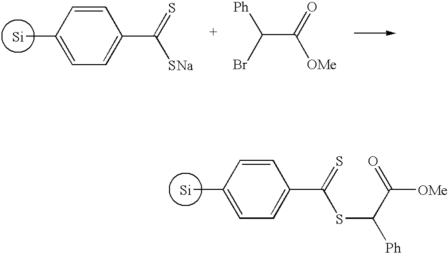

To ethyl acetate (30 cm³) was added bis-thiobenzoate sodium salt functionalised silica (2 g) and methyl α-bromophenyl acetate (0.321 g, 1.42 mmol) and stirred at room temp for 18 h. The solid was filtered off, washed with ethyl acetate (200 cm³) and diethyl ether (200 cm³) and dried to yield a pale pink solid. Elemental analysis: C, 8.85; H, 1.4; S, 2.1. From the sulphur content gives a loading of 0.328 mmol g⁻¹.

### Example 17

### Silica Supported Propanethiol

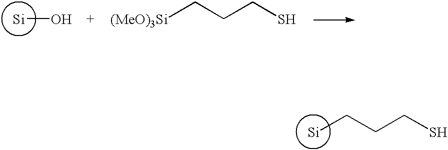

Silica (10 g) and imidazole (0.73 g, 10 mmol) were suspended in DMF (75 cm³) and degassed with nitrogen for 30 mins. To the suspension was added (3-mercaptopropyl)trimethoxysilane (1 cm³, 5.4 mmol) dropwise. The solution was heated to 100° C. under N₂ for 20 h. The solution was cooled and filtered and the silica washed with acetone (500 cm³), toluene (100 cm³) and finally acetone (200 cm³). The silica was the dried under vacuum.

### Silica Supported Propanetrithiocarbonate Sodium Salt

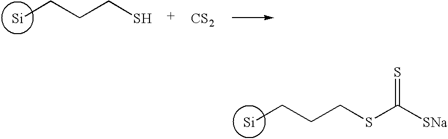

To dioxane (30 cm³) was added silica supported propanethiol (4 g) and finely ground KOH (00.10 g, 1.84 mmol). To the suspension was added CS₂ (0.17 g, 2.21 mmol) dropwise and the mixture stirred at room temperature overnight. To the solution was added diethyl ether (30 cm³) and stirred for 1 h. The silica was filtered off and washed with diethyl ether (150 cm³) then dried under vacuum.

### Silica supported S-methoxycarboyl-α-phenylmethyl propanetrithiocarbonate

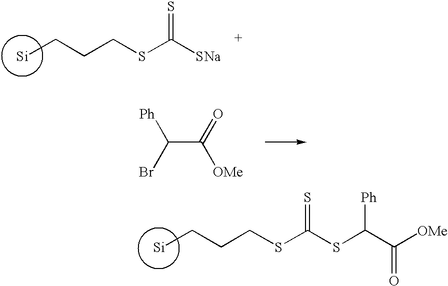

To ethyl acetate (30 cm³) was added trithiopropanoic acid sodium salt functionalised silica (3 g) and methyl α-bromophenyl acetate (0.321 g, 1.42 mmol) and stirred at room temp for 18 h. The silica was filtered off, washed with ethyl acetate (100 cm³) and diethyl ether (2×100 cm³) and dried to yield a pale yellow solid. Elemental analysis: C, 9.45; H, 1.35; S, 3.75. From the sulphur content gives a loading of 0.390 mmol g⁻¹.

### Example 18

### Synthesis of S-cyanoisopropyl trimethoxysilylpropyl trithiocarbonate

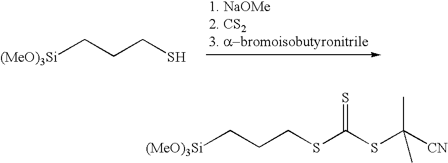

To dry methanol (20 cm³) under nitrogen was added mercaptopropyl trimethoxysilane (3 g, 15.3 mmol). To this was added Sodium Methoxide (0.83 g, 15.3 mmol, 25% sol. in methanol) dropwise and stirred for 5 mins. To the purple solution was added carbon disulphide (1.16 g, 15.3 mmol) dropwise and the solution turned yellow. The solution was stirred for 2 h. To the solution was added α-bromoisobutyronitrile (2.11 g, 15.3 mmol) and stirred for 18 h. The solvent was removed and used without further purification.

### Example 19

### Silica supported S-cyanoisopropyl propyl trithiocarbonate

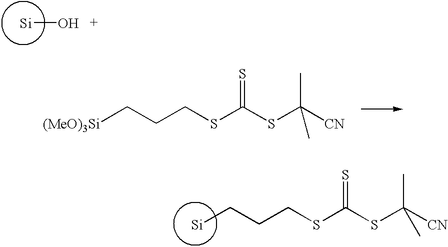

To toluene (25 cm³) was added silica (4 g) and degassed with nitrogen for 30 mins. To the slurry was added S-cyanoisopropyl trimethoxysilylpropyl trithiocarbonate and heated to 80° C. for 2.5 h. The solution was cooled and the solid filtered off, washed with toluene (200 cm³), methanol (200 cm³) and diethyl ether (200 cm³) then dried under vacuum.

### Example 20

### Control Reaction, Polymerisation of Methyl Acrylate with Silica

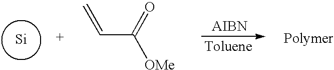

To an ampule was added toluene (90 cm³), methyl acrylate (9.04 g, 105 mmol), AIBN (0.006 g, 0.035 mmol) and silica (1 g) and degassed with N₂ for 10 mins. The solution was heated to 60° C. with stirring for 18 h. The reaction was cooled and THF added to dissolve the polymer. The solution was filtered to remove free polymer and monomer, filtrate analysed by GPC. The silica was washed with toluene (200 cm³), tetrahydrofuran (200 cm³) and acetone (200 cm³) then dried under vacuum.

### Free Polymer Mn=70300 PD=1.57

Polymer cleaved from silica: no polymer observed.

### Example 21

### Polymerisation of methyl acrylate with silica supported S-methoxycarbonyl-α-phenylmethyl dithiobenzoate

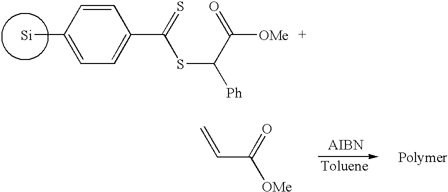

To an ampule was added toluene (10 cm³), methyl acrylate (9.04 g, 105 mmol), AIBN (0.006 g, 0.035 mmol) and silica supported RAFT reagent (1 g, 0.35 mmol) and degassed with nitrogen for 10 mins. The solution was heated to 60° C. with stirring for 18 h. The reaction was cooled and THF added to dissolve the polymer. The solution was filtered to remove free polymer and monomer, filtrate analysed by GPC. The silica was washed with acetone (200 cm³) then dried under vacuum. To cleave the polymer off the silica, the silica was added to toluene (10 cm³) and AIBN (0.57 g, 3.5 mmol) added. The solution was degassed with nitrogen for 10 minutes. The solution was heated to 60° C. for 2 h. The solution was filtered off and the filtrate evaporated to yield a white solid, solid analysed by GPC.

### Free Polymer Mn=263000 PD=1.80; Mn=58700 PD=1.65

Polymer cleaved from silica: Mn=817 PD=1.07; Mn=835 PD=1.06

### Example 22

### Polymerisation of methyl acrylate with silica supported S-methoxycarbonyl-α-phenylmethyl propanetrithiocarbonate

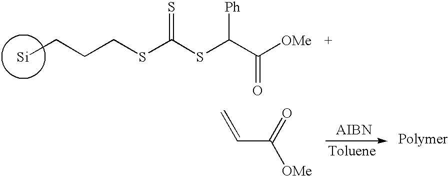

To an ampule was added toluene (90 cm³), methyl acrylate (9.04 g, 105 mmol), AIBN (0.006 g, 0.035 mmol) and silica supported RAFT reagent (1 g, 0.46 mmol) and degassed with nitrogen for 10 mins. The solution was heated to 60° C. with stirring for 18 h. The reaction was cooled and THF added to dissolve the polymer. The solution was filtered to remove free polymer and monomer, filtrate analysed by GPC. The silica was washed with toluene (100 cm³), tetrahydrofuran (100 cm³) and acetone (200 cm³) then dried under vacuum. To cleave the polymer off the silica, the silica (0.5 g) was added to toluene (10 cm³) and AIBN (0.378 g, 2.3 mmol) added. The solution was degassed with nitrogen for 10 minutes. The solution was heated to 60° C. for 2 h. The solution was filtered off and the silica washed with toluene (100 cm³) and tetrahydrofuran (100 cm³) and the filtrate evaporated to yield a white solid. Solid analysed by GPC.

### Example 23

### Polymerisation of methyl acrylate with silica supported S-methoxycarbonyl-α-phenylmethyl dithiobenzoate and free S-methoxycarbonyl-α-phenylmethyl dithiobenzoate

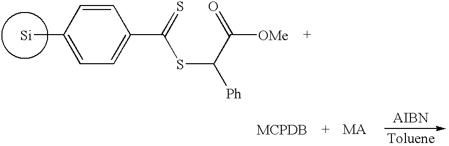

To an ampule was added toluene (25 cm³), methyl acrylate (3.36 g, 39 mmol), AIBN (0.0013 g, 7.8 μmol), Silica-MCPDB (1 g, 0.078 mmol) and MCPDB (0.012 g, 0.039 mmol) then degassed with nitrogen for 10 mins. The solution was heated to 60° C. with stirring for 24 h. The reaction was cooled and the silica filtered off and washed with toluene (100 cm³), tetrahydrofuran (200 cm³) the hot tetrahydrofuran (100 cm³) then dried under vacuum. The filtrate was characterised by GPC analysis. To cleave the polymer off the silica, the silica was added to toluene (10 cm³) and AIBN (0.13 g, 0.78 mmol) added. The solution was degassed with nitrogen for 10 minutes. The solution was heated to 60° C. for 2 h. The solution was filtered off and the filtrate evaporated to yield a white solid. The filtrate was characterised by GPC analysis.

### Free Polymer Mn=16600 PD=1.32

Polymer cleaved from silica: Mn=77200 PD=1.12

### Example 24

### Preparation of 3-(Benzylthiocarbonylsulanyl)-propionic acid

### Method 1

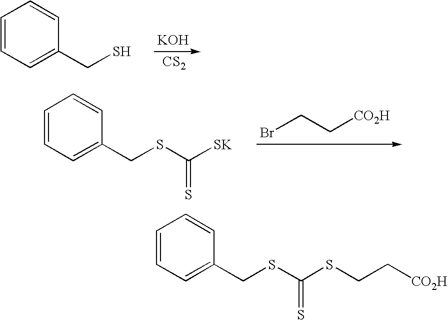

To a solution of KOH (2.26 g, 40 mmol) in water (100 cm³) was added benzyl mercaptan (5 g, 40 mmol) followed by carbon disulphide (2.45 cm³, 40 mmol) and stirred for 5 h. To the orange solution was added 3-bromopropionic acid (6.16 g, 40 mmol) and heated to 80° C. for 12 h. The solution was cooled and extracted with ethyl acetate, dried over MgSO₄ and the solvent removed. The product was purified by column chromatography (ethyl acetate:hexane 3:1) to yield 3.10 g of a yellow solid.

### Method 2

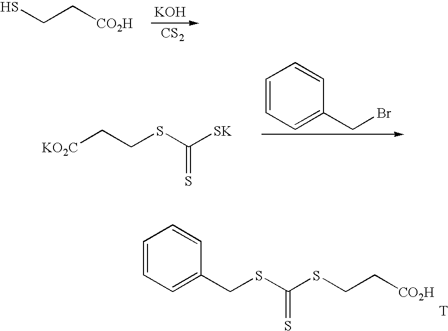

To a solution of KOH (13 g, 231.7 mmol) in water (100 cm³) was added benzyl mercaptopropionic acid (13 cm³) followed by carbon disulphide (15 cm³) and stirred for 5 h. To the orange solution was added benzyl bromide (19.2 g, 116 mmol) and heated to 80° C. for 18 h. The solution was cooled and extracted with ethyl acetate, dried over MgSO₄ and the solvent removed. The product was purified by column chromatography (ethyl acetate:hexane 3:1) to yield 3.10 g of a yellow solid.

### Example 25

### Preparation of S-Methoxycarbonylphenylmethyl 2-hydroxyethyltrithiocarbonate

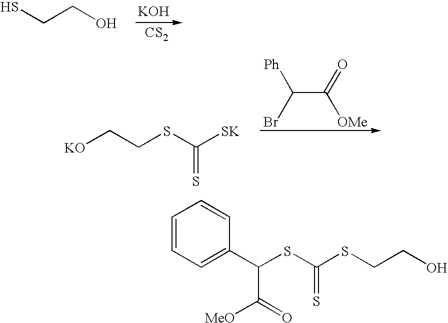

2-Mercaptoethanol (3.44 g, 44 mmol) was transferred to a round bottom flask containing a solution of potassium hydroxide (2.47 g, 44 mmol) in 50 μL of water. The solution was stirred for 10 min and followed by the dropwise addition of carbon disulphide (5.75 mL). The orange oil was continuously stirred at an ambient temperature for 5 h. After that methyl-α-bromophenylacetate (10.0 g, 44 mmol) was added to the round bottom flask. The mixture was allowed to cool and dichloromethane (DCM) was added (100 mL, 3 times). The DCM layer was dried over anhydrous magnesium sulphate and the solvent was evaporated by reducing pressure. The orange oily was purified by pass through the silica gel using 7:3 hexane/ethyl acetate as an eluent to afford a yellow liquid.

### Example 26

### Preparation of 3-(Methoxycarbonyl-phenyl-methylsulfanylthiocarbonylsulfanyl)-propionic acid

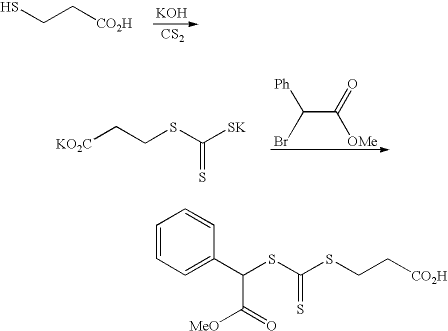

3-Mercaptopropionic acid (4 mL, 46 mmol) was transferred to a round bottom flask containing a solution of potassium hydroxide (5.2 g, 96 mmol) in 50 mL of water. The solution was stirred for 10 min and followed by the dropwise addition of carbon disulphide (6 mL, 62 mmol). The orange oil was stirred for 5 hour. The orange oil was continuously stirred at an ambient temperature for 5 h. After that methyl-α-bromophenylacetate (10.5 g, 46 mmol) was added to the round bottom flask The mixture was allowed to cool and 150 mL of dichloromethane (DCM) was added. Concentrated hydrochloric acid was added to acidify until the organic layer became yellow and the colour in aqueous phase did not change. The water phase was extracted twice with DCM. The DCM layer was dried over anhydrous magnesium sulphate and the solvent was evaporated by reducing pressure. The orange oily was purified by pass through the silica gel using a gradient eluent of 6:1 to 3:1 hexane/ethyl acetate to afford a yellow liquid.

### Example 27

### Merrifield-S-methoxycarbonylphenylmethyl 2-hydroxyethyltrithiocarbonate

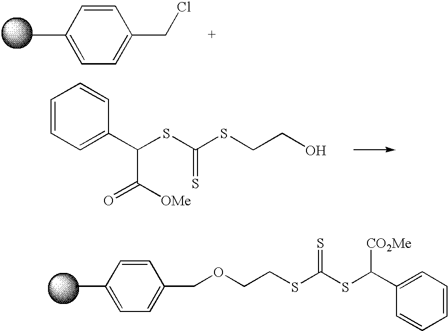

Merrifield resin (3 g, 3 mmol) was placed into a round bottom flask and 150 mL of THF was added. Triethylamine (0.5 g, 5 mmol) was transferred into the flask. The mixture was stirred gently for 5 min. After that MCPHT (1.0 g, 3.3 mmol) was added dropwise and the mixture was allowed to refluxed for 12 h. After cooling the reaction to ambient temperature, the solid was filtered and then washed with THF (100 mL×2), then a mixture of water and THF (1:1) (100 mL×2), H₂O (100 mL×2); acetone (50 mL×2), toluene (50 mL×2) and acetone (50 mL×2). The pale yellow solid was dried over night in vacuum oven.

### Example 28

### Preparation of Merrifield-3-(Methoxycarbonyl-phenyl-methylsulfanylthiocarbonylsulfanyl)-propionic acid

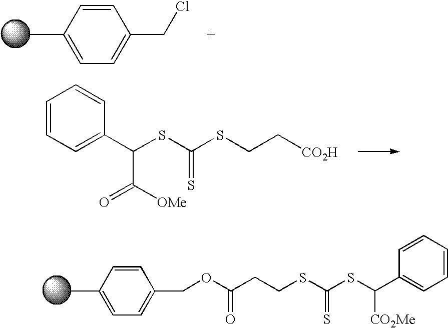

Merrifield resin (2 g, 2 mmol of Cl) was added to a round bottom flask containing 40 mL of THF and potassium carbonate (1.10 g, 8 mmol). The suspension was stirred gently for 5 min at ambient temperature. MCPPA (1.32 g, 4 mmol) was dissolved in 20 mL (×2) of THF in a 50 mL beaker and then transferred to the round bottom flask. Tetra-n-butyl ammonium iodide (1.85 g, 5 mmol) was added to the flask. The temperature was raised to 60° C. and kept at this temperature for 12 h. After cooling the temperature, the solid was filtered and then washed with THF (100 mL×2), then a mixture of water and THF (1:1) (100 mL×2), H₂O (100 mL×2), acetone (50 mL×2), toluene (50 mL×2) and acetone (50 mL×2). The deep yellow solid was dried over night in vacuum oven.

### Example 29

### Preparation of Merrifield-3-(Benzylthiocarbonylsulfanyl)-propionic acid

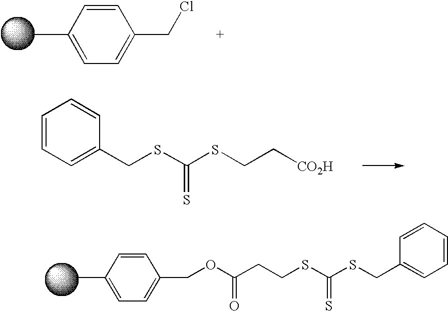

Merrifield resin (2 g, 2 mmol of Cl) was added to a round bottom flask containing 40 mL of THF and potassium carbonate (1.10 g, 8 mmol). The suspension was stirred gently for 5 min at ambient temperature. BSSPA (1.09 g, 4 mmol) was dissolved in 20 mL (×2) of THF in a 50 mL beaker and then transferred to the round bottom flask. Tetra-n-butyl ammonium iodide (1.85 g, 5 mmol) was added to the flask. The temperature was raised to 60° C. and kept at this temperature for 12 h. After cooling the temperature, the solid was filtered and then washed with THF (100 mL×2), then a mixture of water and THF (1:1) (100 mL×2), H₂O (100 mL×2), acetone (50 mL×2), toluene (50 mL×2) and acetone (50 mL×2). The deep yellow solid was dried over night in vacuum oven.

### Example 30

### Preparation of Silica-3-(Benzylthiocarbonylsulfanyl)-propionic acid

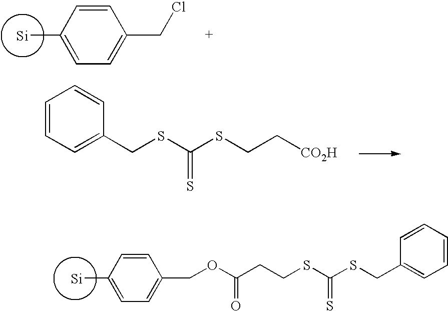

To a suspension of benzyl chloride supported silica (2 g, 3.04 mmol) in acetone (50 cm³) was added 3-Benzylsulfanylthiocarbonylsulfanylpropionic acid (1.65 g, 6.08 mmol) K₂CO₃ (0.84 g, 6.08 mmol) and tetrabutylammonium iodide (1.12 g, 3.04 mmol) and heated to 60° C. for 18 h. The suspension was cooled and the solid filtered off. The silica was washed with acetone (200 cm³), water (200 cm³), acetone (200 cm³), toluene (100 cm³) and diethyl ether (100 cm³) then dried under vacuum to yield a yellow solid.

### Example 31

### Polymerisation of styrene and removal of attached polystyrene using Merrifield-3-(Benzylthiocarbonylsulfanyl)-propionic acid

The Merrifield resin (0.5 g, 0.7 mmol/g) was suspended in a solution of styrene (6.1 g) in toluene (6.1 g) and AIBN (0.004 g) was added (ratio of 100:1:0.2 (monomer:Merrifield resin:initiator)), degassed with nitrogen for 10 mins and heated to 80° C. for 48 h. The solution was cooled, filtered and the Merrifield resin washed with tetrahydrofuran. The filtrate (“free” polymer) was analysed by gel permeation chromatography (GPC). The resin was washed with toluene and tetrahydrofuran until all the “free” polymer had been removed. The dried resin was then suspended in toluene. To the suspension was added AIBN (0.40 g), with a ratio of 10:1 (AIBN:Merrifield resin), degassed for 10 mins and heated to 80° C. for 2.5 h. The solution was cooled, filtered and the Merrifield resin washed with tetrahydrofuran. The filtrate (“attached” Polymer) was analysed by gel permeation chromatography (GPC).

### Reaction 1

### “Free” Polymer Mn=29680 PD=1.31

### “Attached” Polymer Mn=1530 PD=1.18

### Reaction 2

### “Free” Polymer Mn=22520 PD=1.37

### “Attached” Polymer Mn=1340 PD=1.21

### Reaction 3 (Polymerisation Time Increased to 96 h)

### “Free” Polymer Mn=24100 PD=1.32

### “Attached” Polymer Mn=1700 PD=1.20

### Example 32

### Polymerisation of styrene and removal of attached polystyrene using Merrifield-3-(Benzylthiocarbonylsulfanyl)-propionic acid with added “free” 3-(Benzylthiocarbonylsulfanyl)-propionic acid

The Merrifield resin (0.5 g, 0.65 mmol/g) was suspended in a solution of styrene (8.49 g) in toluene (8.49 g). To the suspension was added AIBN (0.005 g) and “free” 3-(Benzylthiocarbonylsulfanyl)-propionic acid (see below for ratios), degassed with nitrogen for 10 mins and heated to 60° C. for 48 h. The solution was cooled, filtered and the Merrifield resin washed with tetrahydrofuran. The filtrate (“free” polymer) was analysed by gel permeation chromatography (GPC). The resin was washed with toluene and tetrahydrofuran until all the “free” polymer had been removed. The dried resin was then suspended in toluene. To the suspension was added AIBN (1.07 g), with a ratio of 20:1 (AIBN:Merrifield resin), degassed for 10 mins and heated to 80° C. for 2.5 h. The solution was cooled, filtered and the Merrifield resin washed with tetrahydrofuran. The filtrate (“attached” Polymer) was analysed by gel permeation chromatography (GPC).

### Reaction 1—Ratio 250:1:1:0.1 (Monomer:Merrifield Resin:Free-CTA:Initiator)

### “Free” Polymer Mn=8300 PD=1.31

### “Attached” Polymer Mn=2650 PD=1.36

### Reaction 2—Ratio 250:1:0.5:0.1 (Monomer:Merrifield Resin:Free-CTA:Initiator)

### “Free” Polymer Mn=8700 PD=1.28

### “Attached” Polymer Mn=2450 PD=1.40

### Reaction 3—Ratio 250:1:0.25:0.1 (Monomer:Merrifield Resin:Free-CTA:Initiator)

### “Free” Polymer Mn=7100 PD=1.30

“Attached” Polymer Mn=2200 PD=1.32

1. A method of making a functionalised polymer of Formula (1) or Formula (2). 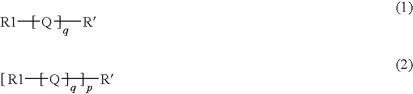
comprising the steps of:
reacting a thiocarbonyl thio compound of Formula (3) or Formula (4); 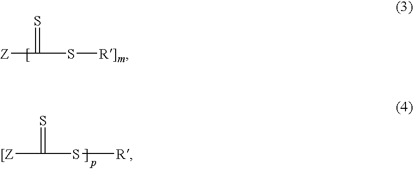
an olefinically unsaturated monomer (Q), and a first source of free radical to form a Polymer of Formula (6) or Formula (7); 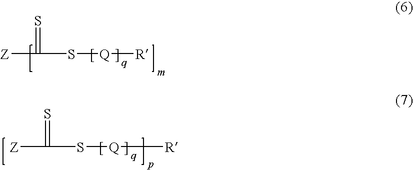
and subsequently contacting the polymer of Formula (6) or Formula (7) with a second source of free radicals, the second source of free radicals comprising a radically transferable functional moiety R1, to form a polymer of Formula (1) or Formula (2) and a compound of Formula (3) or Formula (4);
wherein:
R1 is moiety comprising a functional group;
R′ is selected from the group consisting of alkyl, substituted alkyl, alkoxy, substituted alkoxy, an aromatic saturated or unsaturated carbocyclic or heterocyclic ring, optionally substituted with one or more substituents, amino alkyl, cyanoalkyl, hydroxylalkyl, saturated and unsaturated amido; an organometallic species, a polymer chain and any of the foregoing substituted with one or more CN or OH groups;
Z is selected from (i) a solid support, (ii) Z comprises a linker attached to a solid support, and (iii) Z is a group selected from a straight or branched chain, substituted or non substituted C₁ to C₂₀ alkyl (especially a C₁ to C₄ alkyl such as methyl or ethyl); optionally substituted aryl, e.g. phenyl, substituted phenyl; phenyl covalently bonded to a polymer; optionally substituted heterocyclyl, substituted or non-substituted C₁ to C₂₀ (especially C₁ to C₄) alkoxy, optionally substituted alkyl thio, thioalkoxyl (optionally substituted with a polymer); substituted or non-substituted benzyl (optionally substituted with a solid support), optionally substituted aryl oxycarbonyl (—COOR″), carboxy (—COOH), optionally substituted ocyloxy (—O₂CR″), optionally substituted acyloxy (—CO₂CR″), optionally substituted carbornyl (—CONR″₂), cyano (—CN), dialkyl- or diaryl phosphonato (—P(═OR″Z), dialkyl- or diaryl-phosphinato [—P(═O)R″Z] or SCH₂CH₂CO₂T (where T is a solid support or a polymer); the linker may optionally comprise a straight or branched chain, substituted or non substituted C₁ to C₂₀ alkyl (especially a C₁ to C₄ alkyl such as methyl or ethyl); phenyl, substituted phenyl; phenyl covalently banded to a polymer; substituted or non-substituted C₁ to C₂₀ (especially C₁ to C₄) alkoxy, thioalkoxyl (optionally substituted with a polymer); substituted or non-substituted benzyl;
most preferably Z is a solid support or a linker attached to a solid support;
R″ is selected from the group consisting of optionally substituted C₁-C₁₈ alkyl, C₂-C₁₈ alkenyl, aryl, heterocyclyl, aralkyl, alkaryl wherein the substituents are independently selected from the group that consists of epoxy, hydroxyl, alkoxy, acyl, acyloxy, carboxy (and salts), sulfonic acid (and salts), alkoxy- or aryloxycarbonyl, isocyanato, cyano, silyl. halo, and dialkylamino;
Q is at least one olefinically unsaturated monomer, optionally two or more different olefinically unsaturated monomers;
q=an integer of at least 2;
p=an integer of at least 1;
m=an integer of at least 1.

2. A method of making a functionalised polymer according to claim 1, wherein the olefinically unsaturated monomer comprises vinyl monomers of Formula (5): 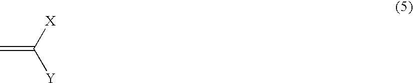
wherein X is selected from the group consisting of: hydrogen, halogen and substituted or unsubstituted C₁-C₄ alkyl, said alkyl substituents being independently selected for the group consisting of hydroxyl, alkoxy, OR″, CO₂H, CO₂R″, O₂CR″ and combinations thereof; and
wherein Y is selected from the group consisting of hydrogen, R″, CO₂H, CO₂R″, COR″, CN, CONH₂, CONHR″, CONR″₂, O₂CR″, OR″ and halogen.

3. A method as claimed in claim 1 wherein the compound of Formula (3) or (4) is recovered at the end of the process.

4. A method as claimed in claim 1 wherein the second source of radicals is a compound capable of forming a carbon or oxygen centred radical of Formula (8)
R2-W—R3
Wherein R2 and R3 are independently selected from the group R′; and
W is a N═N bond, an O—O bond or a group that decomposes thermally or photolytically to form two residues containing a carbon or oxygen centred radical and at least one of R2 or R3 reacts with the polymer of Formula (6) or Formula (7) to leave the moiety R1 comprising the functional group.

5. A method according to claim 4, wherein R1, R2 and/or R3 may be the same or different and are selected from a group consisting of alkyl, substituted alkyl, alkoxy, substituted alkoxy, an aromatic saturated or unsaturated carbocyclic or heterocyclic ring, optionally substituted with one or more substituents, amino alkyl, cyanoalkyl, hydroxylalkyl, saturated and unsaturated amido; an organometallic species, a polymer chain and any of the foregoing substituted with one or more CN or OH groups.

6. A method as claimed in claim 1 wherein the group Z is selected from the group consisting of: methyl, ethyl, other C₁-C₄ alkyl, methylene covalently bonded to a polymer, methylene covalently bonded to a solid support T, phenyl, substituted phenyl, phenyl covalently bonded to a polymer, phenyl covalently bonded to solid support T, alkoxy, substituted alkoxy, thioalkoxy, substituted with a solid support T, benzyl, substituted benzyl, benzyl substituted with a polymer, benzyl substituted with a solid support T, SCH₂.CH₂.CO₂T wherein T is a polymer or solid support and preferably SCH₂.CH₂.CO₂T wherein T is a solid support or polymer.

7. A method as claimed in claim 6 wherein the group Z is selected from the group consisting of: 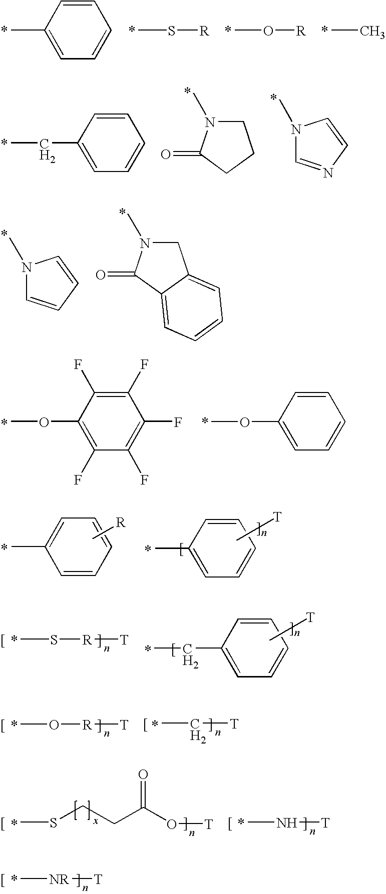
wherein T is a solid support selected from an organic compound, an inorganic compound or magnetised beads;
R is selected from a group consisting of alkyl, substituted alkyl, alkoxy, substituted alkoxy, an aromatic saturated or unsaturated carbocyclic or heterocyclic ring, optionally substituted with one or more substituents, amino alkyl, cyanoalkyl, hydroxylalkyl, saturated and unsaturated amido; an organometallic species, a polymer chain and any of the foregoing substituted with one or more CN or OH groups;
n=an integer of at least 1
x=an integer greater than 1.

8. A method as claimed in claim 1 wherein the group R′ and/or R1 is selected from the group consisting of:

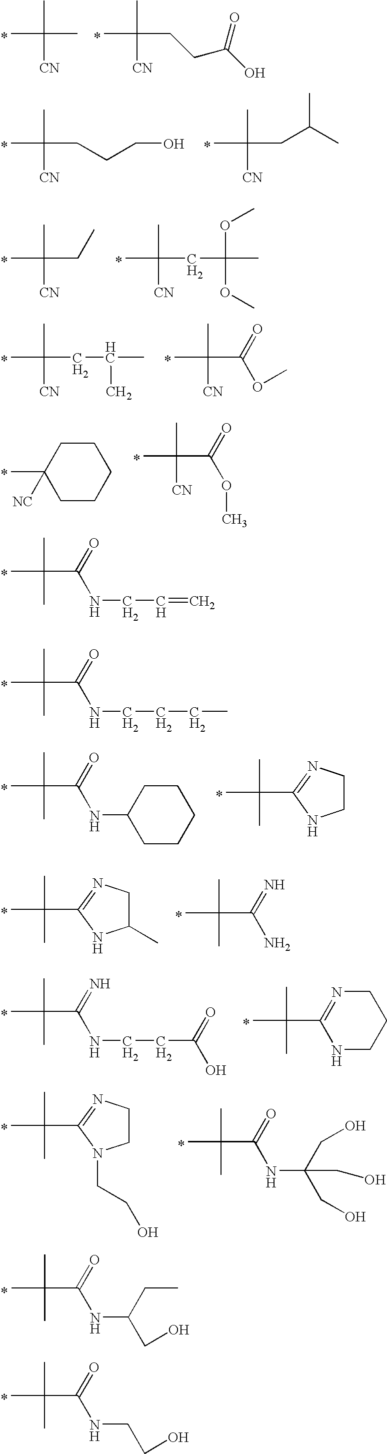

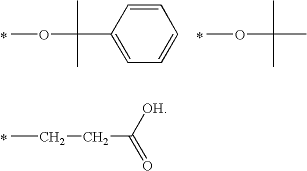

9. A method as claimed in claim 1 wherein the olefinically unsaturated monomer or comonomers are selected from the group consisting of: methyl methacrylate, ethyl acrylate, propyl methacrylate (all isomers), butyl methacrylate (all isomers), 2-ethylhexyl methacrylate, isobornyl methacrylate, methacrylic acid, benzyl methacrylate, phenyl methacrylate, methacrylonitrile, alpha-methylstyrene, methyl acrylate, ethyl acrylate, propyl acrylate (all isomers), butyl acrylate (all isomers), 2-ethylhexyl acrylate, isobornyl acrylate, acrylic acid, benzyl acrylate, phenyl acrylate, acrylonitrile, styrene, acrylates and styrenes selected from glycidyl methacrylate, 2-hydroxyethyl methacrylate, hydroxypropyl methacrylate (all isomers), hydroxybutyl methacrylate (all isomers), N,N-dimethylaminoethyl methacrylate, N,N-diethylaminoethyl methacrylate, triethyleneglycol methacrylate, itaconic anhydride, itaconic acid, glycidyl acrylate, 2-hydroxyethyl acrylate, hydroxypropyl acrylate (all isomers), hydroxybutyl acrylate (all isomers), N,N-dimethylaminoethyl acrylate, N,N-diethylaminoethyl acrylate, triethyleneglycol acrylate, methacrylamide, N-methylacrylamide, N,N-dimethylacrylamide, N-tert-butylmethacrylamide, N-n-butylmethacrylamide, N-methylolacrylamide, N-ethylolacrylamide, vinyl benzoic acid (all isomers), diethylaminostyrene (all isomers), alpha-methylvinyl benzoic acid (all isomers), diethylamino alpha-methylstyrene (all isomers), p-vinylbenzenesulfonic acid, p-vinylbenzene sulfonic sodium salt, trimethoxysilylpropyl methacrylate, triethoxysilylpropyl methacrylate, tributoxysilylpropyl methacrylate, dimethoxymethylsilylpropyl methacrylate, diethoxymethylsilylpropylmethacrylate, dibutoxymethylsilylpropyl methacrylate, diisopropoxymethylsilylpropyl methacrylate, dimethoxysilylpropyl methacrylate, diethoxysilylpropyl methacrylate, dibutoxysilylpropyl methacrylate, diisopropoxysilylpropyl methacrylate, trimethoxysilylpropyl acrylate, triethoxysilylpropyl acrylate, tributoxysilylpropyl acrylate, dimethoxymethylsilylpropyl acrylate, diethoxymethylsilylpropyl acrylate, dibutoxymethylsilylpropyl acrylate, diisopropoxymethylsilylpropyl acrylate, dimethoxysilylpropyl acrylate, diethoxysilylpropyl acrylate, dibutoxysilylpropyl acrylate, diisopropoxysilylpropyl acrylate, vinyl acetate, vinyl butyrate, vinyl benzoate, vinyl chloride, vinyl fluoride, vinyl bromide, maleic anhydride, N-phenylmaleimide, N-butylmaleimide, N-vinylpyrrolidone, N-vinylcarbazole, butadiene, isoprene, chloroprene, ethylene, propylene, 1,5-hexadienes, 1,4-hexadienes, 1,3-butadienes, and 1,4-pentadienes.

10. A method as claimed in claim 1 wherein the at least one olefinically unsaturated monomer is selected from the group consisting of: vinyl acetate, N-vinyl formamide, N-alkylvinylamine, allylamine, N-alkylallylamine, diallylamine, N-alkyldiallylamine, alkylenimine, acrylic acids, alkylacrylates, acrylamides, methacrylic acids, alkylmethacrylates, methacrylamides, N-alkylacrylamides, N-alkylmethacrylamides, styrene, vinylnaphthalene, vinyl pyridine, ethylvinylbenzene, aminostyrene, vinylbiphenyl, vinylanisole, vinylimidazolyl, vinylpyridinyl, dimethylaminomethylstyrene, trimethylammonium ethyl methacrylate, trimethylammonium ethyl acrylate, dimethylamino propylacrylamide, trimethylammonium ethylacrylate, trimethylammonium ethyl methacrylate, trimethylammonium propyl acrylamide, dodecyl acrylate, octadecyl acrylate, and octadecyl methacrylate.

11. A method as claimed in claim 1 wherein the at least one olefinically unsaturated monomer is selected from a group consisting of alkylacrylamides, methacrylamides, acrylamides, styrenes allylamines, allylammonium diallylamines, diallylammoniums, alkylmethacrylates, alkylacrylates, methacrylates, acrylates, n-vinyl formamide, vinyl ethers, vinyl sulfonate, acrylic acid, sulfobetaines, carboxybetaines, phosphobetaines, and maleic anhydride.

12. A method as claimed in claim 1 wherein at least one olefinically unsaturated monomer is selected from the group consisting of: alkylmethacrylates, alkylacrylates, methacrylates, acrylates, alkylacrylamides, methacrylamides, acrylamides, and styrenes.

13. A method as claimed in claim 1 wherein the first source of free radical is selected from the group consisting of: 2,2′-azobis(isobutyronitrile), 4,4′-azobis(4-cyanopentanoic acid, 2-(t-butylazo)-2-cyanopropane, 2,2′-azobis(isobutyramide) dihydrate, 2,2′-azobis(2-methylpropane), 2,2′-Azobis[2-(5-methyl-2-imidazolin-2-yl)propane]dihydrochloride, 2,2′-Azobis[2-(2-imidazolin-2-yl)propane]dihydrochloride, 2,2′-Azobis[2-(2-imidazolin-2-yl)propane]disulfate dehydrate, 2,2′-Azobis(2-methylpropionamide)dihydrochloride, 2,2′-Azobis[N-(2-carboxyethyl)-2-methylpropionamidine]tetrahydrate, 2,2′-Azobis[2-(3,4,5,6-tetrahydropyrimidin-2-yl)propane]dihydrochloride, 2,2′-Azobis{2-[1-(2-hydroxyethyl)-2-imidazolin-2-yl]propane}dihydrochloride, 2,2′-Azobis[2-(2-imidazolin-2-yl)propane], 2,2′-Azobis{2-methyl-N-[1,1-bis(hydroxymethyl)-2-hydroxyethyl]propionamide, 2,2′-Azobis{2-methyl-N-[2-(1-hydroxybuthyl)]propionamide}, 2,2′-Azobis[2-methyl-N-(2-hydroxyethyl)propionamide], 2,2′-Azobis(4-methoxy-2,4-dimethyl valeronitrile), 2,2′-Azobis(2,4-dimethyl valeronitrile), Dimethyl 2,2′-azobis(2-methylpropionate), 2,2′-Azobis(2-methylbutyronitrile), 1,1′-Azobis(cyclohexane-1-carbonitrile), 2,2′-Azobis[N-(2-propenyl)-2-methylpropionamide], 1-[(cyano-1-methylethyl)azo]formamide, 2,2′-Azobis(N-butyl-2-methylpropionamide), 2,2′-Azobis(N-cyclohexyl-2-methylpropionamide), t-butyl peroxyacetate, t-butyl peroxybenzoate, t-butyl peroxyoctoate, t-butyl peroxyneodecanoate, t-butylperoxy isobutyrate, t-amyl peroxypivalate, t-butyl peroxypivalate, di-isopropyl peroxydicarbonate, dicyclohexyl peroxydicarbonate, dicumyl peroxide, dibenzoyl peroxide, dilauroyl peroxide, potassium peroxydisulfate, ammonium peroxydisulfate, di-t-butyl, hyponitrite, and dicumyl hyponitrite.

14. Method according to claim 1, wherein the second source of free radicals is selected from the group consisting of: 2,2′-azobis(isobutyronitrile), 4,4′-azobis(4-cyanopentanoic acid, 2-(t-butylazo)-2-cyanopropane, 2,2′-azobis(isobutyramide) dihydrate, 2,2′-azobis(2-methylpropane), 2,2′-Azobis[2-(5-methyl-2-imidazolin-2-yl)propane]dihydrochloride, 2,2′-Azobis[2-(2-imidazolin-2-yl)propane]dihydrochloride, 2,2′-Azobis[2-(2-imidazolin-2-yl)propane disulfate dehydrate, 2,2′-Azobis(2-methylpropionamide)dihydrochloride, 2,2′-Azobis[N-(2-carboxyethyl)-2-methylpropionamidine]tetrahydrate, 2,2′-Azobis[2-(3,4,5,6-tetrahydropyrimidin-2-yl)propane]dihydrochloride, 2,2′-Azobis{2-[1-(2-hydroxyethyl)-2-imidazolin-2-yl]propane}dihydrochloride, 2,2′-Azobis[2-(2-imidazolin-2-yl)propane], 2,2′-Azobis{2-methyl-N-[1,1-bis(hydroxymethyl)-2-hydroxyethyl]propionamide, 2,2′-Azobis{2-methyl-N-[2-(1-hydroxybuthyl)]propionamide}, 2,2′-Azobis[2-methyl-N-(2-hydroxyethyl)propionamide], 2,2′-Azobis(4-methoxy-2,4-dimethyl valeronitrile), 2,2′-Azobis(2,4-dimethyl valeronitrile), Dimethyl 2,2′-azobis(2-methylpropionate), 2,2′-Azobis(2-methylbutyronitrile), 1,1′-Azobis(cyclohexane-1-carbonitrile), 2,2′-Azobis[N-(2-propenyl)-2-methylpropionamide], 1-[(cyano-1-methylethyl)azo]formamide, 2,2′-Azobis(N-butyl-2-methylpropionamide), 2,2′-Azobis(N-cyclohexyl-2-methylpropionamide), t-butyl peroxyacetate, t-butyl peroxybenzoate, t-butyl peroxyoctoate, t-butyl peroxyneodecanoate, t-butylperoxy isobutyrate, t-amyl peroxypivalate, t-butyl peroxypivalate, di-isopropyl peroxydicarbonate, dicyclohexyl peroxydicarbonate, dicumyl peroxide, dibenzoyl peroxide, dilauroyl peroxide, potassium peroxydisulfate, ammonium peroxydisulfate, di-t-butyl, hyponitrite, and dicumyl hyponitrite.

15. A method as claimed in claim 1 when the reaction is carried out in a solvent selected from the group consisting of: water, alcohol, tetrahydrofuran dimethyl sulfoxide, dimethylformamide, acetone, acetonitrile, benzene, toluene and mixtures thereof.

16. A method as claimed in claim 1 wherein a reaction is carried out at a temperature in the range of −20 to +200° C.

17. A method as claimed in claim 16 wherein the reaction is carried out at a temperature in the range of 20 to 150° C.

18. A method as claimed in claim 17 wherein the reaction is carried out at a temperature in the range of 20 to 120° C.

19. A method as claimed in claim 18 wherein the reaction is carried out at a temperature in the range of 60 to 90° C.

20. A method according to claim 1 comprising the step of reacting a first supported thiocarbonyl thio compound of Formula (3) or Formula (4) with the olefinically unsaturated monomer (Q) and the first source of free radical to form a polymer of Formula (6) or Formula (7) in the presence of a second non-supported thiocarbonyl compound, and the first and second thiocarbonyl having identical groups R′.

21. A method of carrying out a reversible-addition-fragmentation chain transfer (RAFT) polymerisation comprising the steps of reacting olefinically unsaturated monomers with a first supported chain transfer agent, in the presence of a second unsupported chain transfer agent, in the presence of a free radical source, to form a polymer.

22. A method according to claim 21 comprising a greater concentration of supported compound than non-supported compound.

23. A method of producing a block copolymer comprising reacting a first unsaturated monomer by a method according to claim 1, wherein the thiocarbonyl thio compound of Formula (3) is supported on a solid support, recovering polymer attached to the solid support, and then reacting the recovered polymer by the method of claim 1 with a second unsaturated monomer to form a block copolymer.

24. A compound for use in a method according to claim 1 comprising the formula: 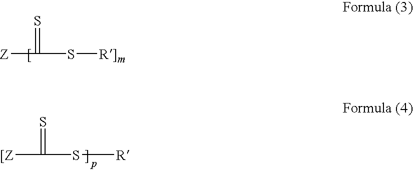
where:
Z is a solid support or a solid support attached via a linker to the thiocarbonyl thio moiety,
m=an integer of at least 1,
p=an integer of at least 1,
R′ is selected from the group consisting of alkyl, substituted alkyl, alkoxy, substituted alkoxy, an aromatic saturated or unsaturated carbocyclic or heterocyclic ring, optionally substituted with one or more substituents, amino alkyl, cyanoalkyl, hydroxylalkyl, saturated and unsaturated amido; an organometallic species, a polymer chain and any of the foregoing substituted with one or more CN or OH groups.

25. A polymer having the formula: 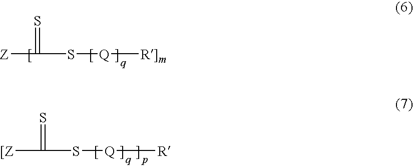
where:
Z is a solid support or a solid support attached via a linker to the thiocarboxylthio moiety,
m=an integer of at least 1,
p=an integer of at least 1,
q=an integer of at least 2,
R′ is selected from the group consisting of alkyl, substituted alkyl, alkoxy, substituted alkoxy, an aromatic saturated or unsaturated carbocyclic or heterocyclic ring, optionally substituted with one or more substituents, amino alkyl, cyanoalkyl, hydroxylalkyl, saturated and unsaturated amido; an organometallic species, a polymer chain and any of the foregoing substituted with one or more CN or OH groups,
Q is at least one olefinically unsaturated monomer, optionally two or more different olefinically unsaturated monomers.

26. A compound or polymer according to claim 24,
wherein Z is selected from: 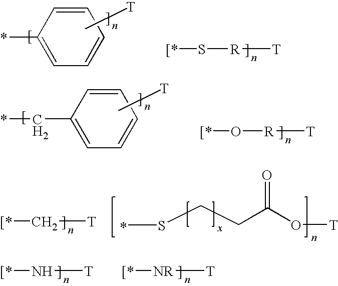
wherein T is a solid support selected from an organic compound, an inorganic compound or magnetised beads,
R is selected from a group consisting of alkyl, substituted alkyl, alkoxy, substituted alkoxy, an aromatic saturated or unsaturated carbocyclic or heterocyclic ring, optionally substituted with one or more substituents, amino alkyl, cyanoalkyl, hydroxylalkyl, saturated and unsaturated amido; an organometallic species, a polymer chain and any of the foregoing substituted with one or more CN or OH groups,
n=an integer of at least 1.

27. A polymer obtainable by a method according to claim 1.

28. A method as claimed in claim 4 wherein the group R2 and/or R3 is selected from the group consisting of:

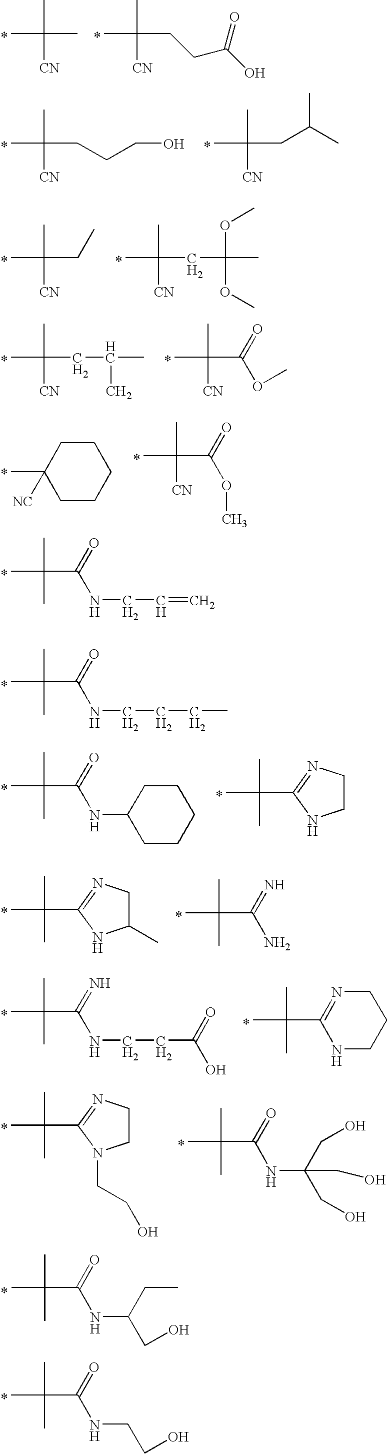

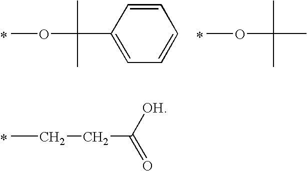

29. A method as claimed in claim 6 wherein the group R is selected from the group consisting of:

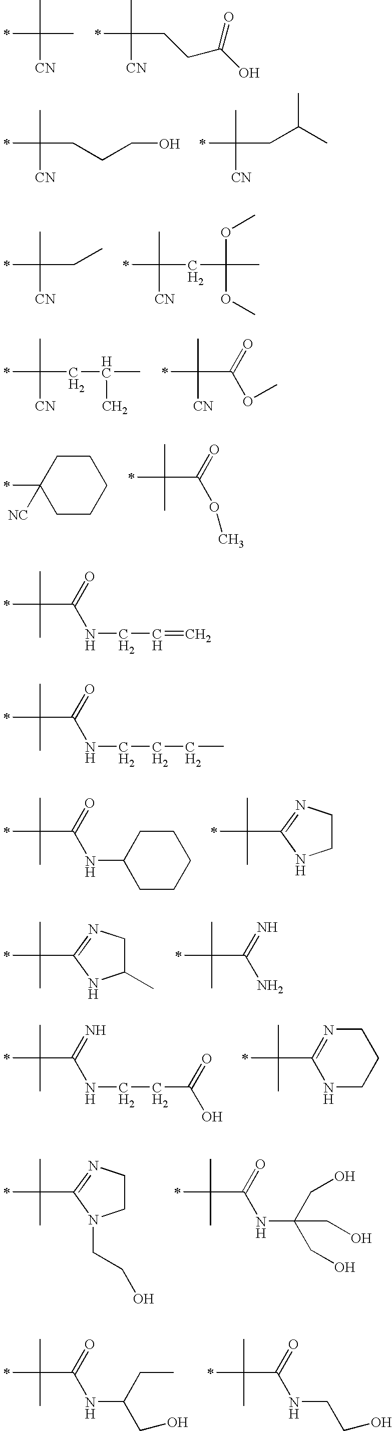

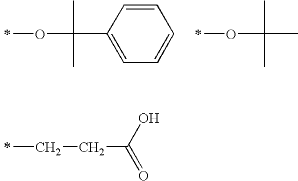

30. A compound or polymer according to claim 25,
wherein Z is selected from: 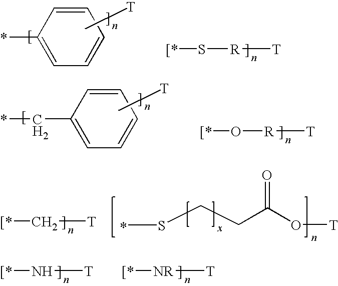
wherein T is a solid support selected from an organic compound, an inorganic compound or magnetised beads,
R is selected from a group consisting of alkyl, substituted alkyl, alkoxy, substituted alkoxy, an aromatic saturated or unsaturated carbocyclic or heterocyclic ring, optionally substituted with one or more substituents, amino alkyl, cyanoalkyl, hydroxylalkyl, saturated and unsaturated amido; an organometallic species, a polymer chain and any of the foregoing substituted with one or more CN or OH groups,
n=an integer of at least 1.
